(19) United States
(12) Patent Application Publication
(10) Pub. No.: US 2026/0042821 A1
(43) Pub. Date: Feb. 12, 2026

(54) HUMAN METAPNEUMOVIRUS ANTIBODIES AND USES THEREOF

(71) Applicant: Fred Hutchinson Cancer Center, Seattle, WA (US)

(72) Inventor: Jim Boonyaratanakornkit, Seattle, WA (US)

(73) Assignee: Fred Hutchinson Cancer Center, Seattle, WA (US)

(21) Appl. No.: 19/119,070

(22) PCT Filed: Oct. 6, 2023
(86) PCT No.: PCT/US2023/076283

§ 371 Date: Apr. 7, 2025

Related U.S. Application Data
(60) Provisional application No. 63/378,727, filed on Oct. 7, 2022.

(51) Int. Cl. C07K 16/10

(52) U.S. Cl. CPC C07K 16/10; C07K 2317/14; C07K 2317/52; C07K 2317/565

(57) ABSTRACT

Antibodies that bind and can neutralize human metapneumovirus (HMPV) are described. The antibodies include antibody 4E11, 3B5, 4F11, and 7F8. Each of these antibodies and binding fragments thereof can be engineered into numerous formats for research, detection, and/or treatment of HMPV, individually and in various combinations.

### CROSS-REFERENCE TO RELATED APPLICATION

This application is a U.S. National Phase Patent Application based on International Patent Application No. PCT/US2023/076283, filed on Oct. 6, 2023, which claims priority to U.S. Provisional Patent Application No. 63/378,727 filed on Oct. 7, 2022, both of which are [[is]] incorporated herein by reference in their [[its]] entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3EF2537.XML. The file is 87,235 bytes, was created Mar. 21, 2025, and is being submitted electronically via Patent Center FIELD OF THE DISCLOSURE

The current disclosure provides antibodies that protect against human metapneumovirus (HMPV). The antibodies and binding fragments thereof can neutralize HMPV and can be engineered into numerous formats for research, detection, and/or treatment of HMPV.

### BACKGROUND OF THE DISCLOSURE

Human metapneumovirus (HMPV) is a respiratory pathogen known to be a major global cause of serious respiratory disease in young children, the elderly, and immunocompromised individuals. Clinically, HMPV respiratory disease is highly analogous to that caused by human respiratory syncytial virus (RSV), and the two pathogens are closely related.

Primary exposure to HMPV typically occurs before age 5 with 90 to 100% of children being infected by HMPV by the age of 5 to 10 years old according to seroprevalence studies. Five to 10% of pediatric hospitalizations are a result of HMPV causing acute lower respiratory tract infections. On average, children who are less than 6 months of age with HMPV infection were three times as likely to be hospitalized compared to children between the ages of 6 months to 5 years.

HMPV reinfections can occur throughout life and can cause severe illness in certain patient populations including the immunocompromised, the elderly, or patients with a pre-existing cardiac or respiratory conditions, with hospitalization rates comparable to RSV and influenza. Further, HMPV has been suggested as a co-pathogen in a subset of severe acute respiratory syndromes caused by the SARS coronavirus, and as a cofactor for pathogenesis in the case of fatal encephalitis.

HMPV is within the Metapneumovirus genus of the family Paramyxoviridae and order Mononegavirales. Similar to RSV, HMPV encodes three surface glycoproteins: the attachment (G), small hydrophobic (SH), and the fusion (F) glycoproteins. The F-glycoprotein promotes fusion of the viral envelope membrane with the host cell membrane, thus, facilitating access of the viral RNA into the target cell cytoplasm. The HMPV F-protein is thus considered to be a major antigenic determinant that mediates effective neutralization and protection against HMPV infection.

### SUMMARY OF THE DISCLOSURE

The current disclosure provides new antibodies that are protective against human metapneumovirus (HMPV). The antibodies and binding fragments thereof can neutralize HMPV and can be engineered into numerous formats for research, detection, and/or treatment of HMPV.

In particular embodiments, the disclosed HMPV antibodies include 4E11, 3B5, 4F11, or 7F8 monoclonal antibodies. In particular embodiments, the HMPV antibody includes
a variable heavy chain including the sequence as set forth in SEQ ID NO: 6 and a variable light chain including the sequence as set forth in SEQ ID NO: 7;
a variable heavy chain including the sequence as set forth in SEQ ID NO: 16 and a variable light chain including the sequence as set forth in SEQ ID NO: 17;
a variable heavy chain including the sequence as set forth in SEQ ID NO: 25 and a variable light chain including the sequence as set forth in SEQ ID NO: 26; or
a variable heavy chain including the sequence as set forth in SEQ ID NO: 74 and a variable light chain including the sequence as set forth in SEQ ID NO: 75.

### BRIEF DESCRIPTION OF THE SEVERAL VIEWS OF THE DRAWINGS

Some of the drawings submitted herewith may be better understood in color. Applicant considers the color versions of the drawings as part of the original submission and reserves the right to present color images of the drawings in later proceedings.

FIG. 1. Screening of 40 million spleen cells from two donors and 160 million peripheral blood mononuclear cells (PBMCs) from two other donors. The 121 HMPV-binding B cells were sorted and 77% of these B cell culture wells were positive for antibody according to ELISA. Of these antibodies, 3 passed a neutralization screen for HMPV. RSV-B-binding B cells were also sorted but none neutralized.

FIG. 2. B cell isolation from human spleen cells, tonsil cells, and PBMCs from de-identified donors. B cell probes consisted of tetramers of HMPV B2 preF (APC) and RSV A preF (PE). Anti-APC and anti-PE magnetic beads were used for single cell sorting enrichment. B cells were individually sorted into culture with feeder cells expressing IL2, IL21, and CD40L to stimulate antibody production. Culture supernatant was then collected for neutralization assays to HMPV A2. Note that B cell probe selection for HMPV B2-binding B cells with a neutralization screen to HMPV2 yields antibodies that can neutralize both HMPVB2 and A2.

FIG. 3. B cell receptor genes were sequenced, cloned, and expressed as monoclonal IgG1 antibodies.

FIGS. 4A, 4B. (4A) hMPV neutralization by 4E11, 4F11, and 3B5 antibodies. (4B) hMPV neutralization by 4F11 and 7F8 antibodies.

FIG. 5. Biolayer interferometry (BLI) with HMPV preF trimer.

FIG. 6. Competition experiments. 4F11 does not compete with ADI-60126 or SAN32-3 (site 0) or with MxR (site 3).

FIG. 7. Structural analysis using negative stain electron microscopy (nsEM). Size exclusion chromatography (SEC) trace of HMPV F indicated that the trimer was intact. SEC trace after mixing HMPV F with 4F11 indicated presence of the 4F11 Fab bound to a protomer.

FIG. 8. nsEM of Trimer+MxR then 4F11. One Fab of 4F11 bound to the monomer, and no MxR Fabs bound. Based on these results, 4F11 still breaks the trimer even with stabilizing mutations and even when MxR is pre-bound to the trimer.

FIG. 9. Effect of amino acid 185. HMPV F was loaded, and 4F11 was used for association. DS-Cav-Es2 has a mutation at amino acid 185 changing alanine to proline to stabilize HMPV F in the prefusion conformation. 4F11 does not bind to DS-Cav-Es2 unless the proline is reverted back to an alanine. Thus, proline 185 disrupts an interaction with 4F11.

FIG. 10. Model predictions. Binding only occurs with the prefusion conformation and not with the postfusion conformation. Binding occurs near the apex at the interface between protomers. A185P is in the epitope for 4F11. The stabilizing mutations of DSCavEs2 lock the trimer and prevent it from opening.

FIG. 11. Production of HMPV F monomer. The GCNT trimerization domain was deleted. Expression/yield of the monomer was 10 times greater than expression/yield of the trimer. Binding was observed on BLI between the Fab and the monomer. Cryo-EM was performed with the 4F11 Fab and HMPV F monomer. Load HMPV monomer or trimer>Bind to IgG or Fab>Dissociate.

FIG. 12. Cryo-EM performed with the 4F11 Fab and HMPV F monomer at a 4 angstrom resolution. 4F11 complexed with HMPV had a preferred orientation, negatively impacting resolution. Co-complexing with MxR improved this resolution.

FIG. 13. P185 for stabilization. In this model, proline 185 does not appear to interact with 4F11.

FIG. 14. Role of HMPV F glycosylation. The HMPV F monomer was loaded onto the probe, and the 4F11 Fab was used for association. For the HMPV F with short glycans condition, the F protein was produced in GNTI⁻ 293 cells. For the HMPV F with no glycans condition, the F protein was produced in GNTI⁻ 293 cells, followed by EndoH treatment.

FIG. 15. Part of a glycan can be visualized at site 0.

FIG. 16. Experimental design for testing in vivo efficacy. Three groups of five animals each were administered: intranasal 4F11 IgG, intramuscular 4F11 IgG, or intranasal negative control.

FIG. 17. Results of in vivo efficacy testing based on nasal turbinate (left panel) and lung (right panel) samples.

### DETAILED DESCRIPTION

Human metapneumovirus (HMPV) is a respiratory pathogen known to be a major global cause of serious respiratory disease in young children, the elderly, and immunocompromised individuals. Despite this, there are no clinically relevant vaccines or anti-viral therapies approved for the treatment or prevention of HMPV infections.

The current disclosure provides antibodies that are protective against HMPV. In particular embodiments, the HMPV antibody includes a 4E11, 3B5, 4F11, or 7F8 monoclonal antibody. In particular embodiments, the HMPV antibody includes
a variable heavy chain including the sequence as set forth in SEQ ID NO: 6 and a variable light chain including the sequence as set forth in SEQ ID NO: 7;
a variable heavy chain including the sequence as set forth in SEQ ID NO: 16 and a variable light chain including the sequence as set forth in SEQ ID NO: 17;
a variable heavy chain including the sequence as set forth in SEQ ID NO: 25 and a variable light chain including the sequence as set forth in SEQ ID NO: 26; or
a variable heavy chain including the sequence as set forth in SEQ ID NO: 74 and a variable light chain including the sequence as set forth in SEQ ID NO: 75.

In particular embodiments, the HMPV antibody neutralizes HMPV with high potency. High potency refers to a neutralization potency of at least 1.5 ng/mL. In particular embodiments, the neutralization potency of 4E11 can include an IC50 of 2.1 ng/mL whereas the MxR and MPE8 antibodies can have neutralization potencies of 7 ng/mL and 36.8 ng/mL, respectively.

The antibodies and binding fragments thereof disclosed herein can inhibit or neutralize HMPV infection and can be engineered into numerous formats for research, detection, and/or treatment purposes.

Aspects of the current disclosure are now described in more supporting detail as follows: (i) Antibodies; (ii) Multi-Domain Binding Molecules; (iii) Expression of Recombinant Antibodies; (iv) Antibody Conjugates; (v) Recombinant Receptors; (vi) Compositions and Formulations; (vii) Kits; (viii) Methods of Use; (ix) Exemplary Embodiments; and (x) Closing Paragraphs. These headings are provided for organizational purposes only and do not limit the scope or interpretation of the disclosure.

### (i) Antibodies

The present disclosure provides antibodies that bind HMPV. In particular embodiments, the fusion protein of HMPV (fusion, partial; GenBank: AAL35370.1) includes the sequence:

(SEQ ID NO: 100)
LLITPQHGLKESYLEESCSTITEGYLSVLRTGWYTNVFTLEVGDVENLTC
ADGPSLIKTELDLTKSALRELRTVSADQLAREEQIENPRQSRFVLGAIAL
GVATAAAVTAGVAIAKTIRLE.

In particular embodiments, the partial nucleocapsid of HMPV (GenBank: AAL35388.3) includes the sequence:

(SEQ ID NO: 101)
AILNESQYTIKRDVGTTTAVTPSSLQQEITLLCGEILYAKHADYKYAAEI
GIQYISTALGSERVQQILRNSGSEVQVVLTRTYSLGKVKNNKGEDLQMLD
IHGVEKSWVEEIDKEARKTMATLLKESSGNIPQNQRPSAPDTPIILLCVG
ALIFTKLASTIEVGLETTVRRANRVLSDALKRYPRMDIPKIARSFYDLFE
QKVYHRSLFIEYGKALGSSSTGSKAESLFVNIFMQAYGAGQTMLRWGVIA
RSSNNIMLGHVSVQAELKQVTEVYDLVREMGPESGLLHLRQSPKAGLLSL
ANCPNFASVVLGNASGLGIIGMYRGRVPNTELFSAAESYAKSLKESNK.

Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4.

The assignment of amino acids to each domain can be in accordance with Kabat numbering (Kabat et al. (1991), “Sequences of Proteins of Immunological Interest,” 5th Ed. Public Health Service, National Institutes of Health, Bethesda, Md. (“Kabat” numbering scheme)); Chothia (AI-Lazikani et al., (1997) JMB 273, 927-948 (“Chothia” numbering scheme)), Martin (Abinandan et al., Mol Immunol. 45:3832-3839 (2008), “Analysis and improvements to Kabat and structurally correct numbering of antibody variable domains”), Gelfand, Contact (MacCallum et al., J. Mol. Biol. 262:732-745 (1996), “Antibody-antigen interactions: Contact analysis and binding site topography,” J. Mol. Biol. 262, 732-745.” (Contact numbering scheme)), IMGT (Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme)), AHo (Honegger A and Plückthun A, “Yet another numbering scheme for immunoglobulin variable domains: an automatic modeling and analysis tool,” J Mol Biol, 2001 Jun. 8; 309(3):657-70, (AHo numbering scheme)), North (North et al., J Mol Biol. 406(2):228-256 (2011), “A new clustering of antibody CDR loop conformations”), or other numbering schemes. Software programs, such as ABodyBuilder can also be used.

In particular embodiments, a binding domain that binds HMPV includes the 4E11 antibody.

In particular embodiments, the 4E11 antibody includes a variable heavy chain including a CDRH1 including GFSFSNYD (SEQ ID NO: 1), a CDRH2 including VSFNGNTK (SEQ ID NO: 2), and a CDRH3 including ASGYCHGGLCHIQNYFAMDV (SEQ ID NO: 3), and a variable light chain including a CDRL1 including KLGDNY (SEQ ID NO: 4); a CDRL2 including QDN; and a CDRL3 including QTWDSSIAPWV (SEQ ID NO: 5).

In particular embodiments, 4E11 antibody includes a variable heavy chain including a CDRH1 encoded by the sequence GGATTCAGTTTCAGTAACTATGAC (SEQ ID NO: 78), a CDRH2 encoded by the sequence GTTTCTTTTAATGGGAATACAAAA (SEQ ID NO: 79), and a CDRH3 encoded by the sequence GCGTCGGGATATTGTCATGGTGGTCTGTGTCACATACAGAACTACTTCGCTATGGACGTC (SEQ ID NO: 80), and a variable light chain including a CDRL1 encoded by the sequence AAATTGGGGGATAATTAT (SEQ ID NO: 81), a CDRL2 encoded by the sequence CAAGATAAC, and a CDRL3 encoded by the sequence CAGACGTGGGACAGCAGCATTGCGCCCTGGGTG (SEQ ID NO: 82).

In particular embodiments, the 4E11 antibody includes a variable heavy chain including the sequence:

(SEQ ID NO: 6)
QVQLVQSGGGVVQPGRSLRLSCAASGFSFSNYDIHWVRQAPGKGLEWVAL
VSFNGNTKYYAESVTGRFTVSRDNSKDTLYLQMNSLRVEDTAVYYCASGY
CHGGLCHIQNYFAMDVWGHGTTVTVSS

and a variable light chain sequence including the sequence:

(SEQ ID NO: 7)
SYELTQPPSVSVSPGQTARITCSRDKLGDNYVSWYQQRPGQSPLLVIFQD
NKRPSGIPARFSGSNSGNTATLTISGTQAMDEADYYCQTWDSSIAPWVFG
GGTKLTVL.

In particular embodiments, the 4E11 antibody includes a variable heavy chain encoded by the sequence:

(SEQ ID NO: 8)
CAGGTGCAGCTGGTGCAGTCCGGGGGAGGGGTGGTCCAGCCTGGGAGGTC
CCTGAGACTCTCTTGTGCAGCCTCTGGATTCAGTTTCAGTAACTATGACA
TACACTGGGTCCGCCAGGCTCCAGGGAAGGGACTGGAGTGGGTGGCTCTT
GTTTCTTTTAATGGGAATACAAAATATTATGCAGAGTCCGTCACAGGCCG
CTTCACCGTCTCCAGAGACAATTCCAAGGACACGTTGTATCTGCAAATGA
ACAGCCTGAGAGTTGAGGACACGGCTGTCTATTATTGTGCGTCGGGATAT
TGTCATGGTGGTCTGTGTCACATACAGAACTACTTCGCTATGGACGTCTG
GGGCCACGGGACCACGGTCACAGTCTCCTCA

and a variable light chain sequence encoded by the sequence:

(SEQ ID NO: 9)
TCCTATGAGCTGACTCAGCCACCCTCAGTGTCCGTGTCCCCAGGACAGAC
AGCCAGAATCACCTGCTCTAGAGATAAATTGGGGGATAATTATGTTTCCT
GGTATCAACAGAGGCCAGGCCAGTCCCCTCTCTTGGTCATCTTTCAAGAT
AACAAGCGACCCTCAGGGATCCCTGCGCGATTTTCTGGCTCCAACTCTGG
GAACACGGCCACTCTGACCATCAGCGGGACCCAGGCTATGGATGAGGCTG
ACTATTACTGTCAGACGTGGGACAGCAGCATTGCGCCCTGGGTGTTCGGC
GGAGGGACCAAGCTGACCGTCCTGG.

In particular embodiments, a binding domain that binds HMPV includes the 3B5 antibody.

In particular embodiments, the 3B5 antibody includes a variable heavy chain including a CDRH1 including GFIVSNNH (SEQ ID NO: 10), a CDRH2 including LYSGGSA (SEQ ID NO: 11), and a CDRH3 including ARDLHAPDKPN (SEQ ID NO: 12), and a variable light chain including a CDRL1 including RGHSTYA (SEQ ID NO: 13); a CDRL2 including VNSDGRH (SEQ ID NO: 14); and a CDRL3 including QTWGTDIHVV (SEQ ID NO: 15).

In particular embodiments, the 3B5 antibody includes a variable heavy chain including a CDRH1 encoded by the sequence GGATTCATCGTCAGTAACAACCAC (SEQ ID NO: 83), a CDRH2 encoded by the sequence CTTTATAGTGGTGGTAGCGCA (SEQ ID NO: 84), and a CDRH3 encoded by the sequence GCGAGAGATCTTCACGCGCCCGACAAACCCAAT (SEQ ID NO: 85), and a variable light chain including a CDRL1 encoded by the sequence AGGGGTCACAGCACCTACGCC (SEQ ID NO: 86), a CDRL2 encoded by the sequence GTTAACAGTGATGGCAGGCAC (SEQ ID NO: 87), and a CDRL3 encoded by the sequence CAGACCTGGGGCACTGACATTCATGTGGTG (SEQ ID NO: 88).

In particular embodiments, the 3B5 antibody includes a variable heavy chain including the sequence:

(SEQ ID NO: 16)
EVQLVESGGGLVQPGGSLRLSCAASGFIVSNNHMTWVRQTPGKGLECVSV
LYSGGSADYADSVKGRFIISRDNSKNTLYLQMNSLRAEDTAVYYCARDLH
APDKPNWGQGTLVTVSS

and a variable light chain sequence including the sequence:

(SEQ ID NO: 17)
QLVLTQSPSASASLGASVKLTCTLSRGHSTYAIAWHQKQPDKGPRYLMRV
NSDGRHTKGGGIPDRFSGSSSGAERYLTISSLQSEDEADYYCQTWGTDIH
VVFGEGTKLTVL.

In particular embodiments, the 3B5 antibody includes a variable heavy chain encoded by the sequence:

(SEQ ID NO: 18)
GAGGTGCAGCTGGTGGAGTCCGGGGGAGGCTTGGTCCAGCCTGGGGGGTC
TCTGAGACTCTCCTGTGCGGCCTCTGGATTCATCGTCAGTAACAACCACA
TGACCTGGGTCCGCCAGACTCCAGGGAAGGGGCTGGAATGTGTCTCGGTT
CTTTATAGTGGTGGTAGCGCAGACTACGCGGACTCCGTGAAGGGCAGATT
CATCATTTCCAGAGACAATTCCAAGAACACCCTGTATCTTCAAATGAACA
GCCTGAGAGCCGAGGACACGGCTGTATATTACTGTGCGAGAGATCTTCAC
GCGCCCGACAAACCCAATTGGGGCCAGGGAACCTTGGTCACCGTCTCCTC
AG

and a variable light chain sequence encoded by the sequence:

(SEQ ID NO: 19)
CAGCTTGTGCTGACTCAATCGCCGTCTGCCTCTGCCTCCCTGGGAGCCTC
GGTCAAGCTCACCTGCACTCTGAGCAGGGGTCACAGCACCTACGCCATCG
CATGGCATCAGAAGCAGCCAGACAAGGGCCCTCGGTACTTGATGAGAGTT
AACAGTGATGGCAGGCACACCAAGGGGGGCGGGATCCCTGATCGCTTCTC
AGGCTCCAGCTCTGGGGCTGAGCGCTACCTCACCATCTCCAGCCTCCAGT
CTGAGGATGAGGCTGACTATTACTGTCAGACCTGGGGCACTGACATTCAT
GTGGTGTTCGGCGAAGGGACCAAGCTGACCGTCCTAG.

In particular embodiments, a binding domain that binds HMPV includes the 4F11 antibody.

In particular embodiments, the 4F11 antibody includes a variable heavy chain including a CDRH1 including GYKFATYW (SEQ ID NO: 20), a CDRH2 including IYPDDSDT (SEQ ID NO: 21), and a CDRH3 including ARCYDFWSGYQFGMDV (SEQ ID NO: 22), and a variable light chain including a CDRL1 including QSLRHDNGYNY (SEQ ID NO: 23); a CDRL2 including LGS, and a CDRL3 including MQTLQTLMFT (SEQ ID NO: 24).

In particular embodiments, the 4F11 antibody includes a variable heavy chain including a CDRH1 encoded by the sequence GGATACAAGTTTGCCACCTACTGG (SEQ ID NO: 89), a CDRH2 encoded by the sequence ATCTATCCTGATGACTCTGATACC (SEQ ID NO: 90), and a CDRH3 encoded by the sequence GCGAGATGCTACGATTTTTGGAGTGGTTATCAGTTCGGTATGGACGTC (SEQ ID NO: 91), and a variable light chain including a CDRL1 encoded by the sequence CAGAGCCTCCGGCATGATAATGGATACAACTAT (SEQ ID NO: 92), a CDRL2 encoded by the sequence TTGGGTTCT, and a CDRL3 encoded by the sequence ATGCAAACTCTACAAACTCTGATGTTCACT (SEQ ID NO: 93).

In particular embodiments, the 4F11 antibody includes a variable heavy chain including the sequence:

(SEQ ID NO: 25)
EVQLVQSGAEVKKPGDSLKISCKGSGYKFATYWIGWVRQMPGKGLEWMGV
IYPDDSDTRYSPSFQGQVSISVDKSITTAYLQWSSLKASDTAIYYCARCY
DFWSGYQFGMDVWGQGTTVTVSS

and a variable light chain sequence including the sequence: DIVMTQSPLSLPVTPGETASISCRSSQSLRHDNGYNYLDWYLQKPGQSPQLLIYLGSKRASGV PDRFSGSGSGTDFTLKISRVEAEDVGVYYCMQTLQTLMFTFGGGTKVEIK (SEQ ID NO: 26).

In particular embodiments, the 4F11 antibody includes a variable heavy chain encoded by the sequence:

(SEQ ID NO: 27)
GAGGTGCAGCTGGTGCAGTCTGGAGCAGAGGTGAAAAAGCCCGGTGACTC
TCTGAAGATCTCCTGTAAGGGTTCTGGATACAAGTTTGCCACCTACTGGA
TCGGCTGGGTGCGCCAGATGCCCGGGAAAGGCCTGGAGTGGATGGGGGTC
ATCTATCCTGATGACTCTGATACCAGATACAGCCCGTCCTTCCAAGGCCA
GGTCTCCATCTCAGTCGACAAATCCATCACTACCGCCTACTTGCAGTGGA
GTAGCCTGAAGGCCTCGGACACCGCCATATATTACTGTGCGAGATGCTAC
GATTTTTGGAGTGGTTATCAGTTCGGTATGGACGTCTGGGGCCAGGGGAC
CACGGTCACCGTCTCCTCA

and a variable light chain sequence encoded by the sequence:

(SEQ ID NO 28)
GATATTGTGATGACTCAGTCTCCACTCTCCCTGCCCGTCACTCCTGGAG
AGACGGCCTCCATCTCCTGCAGGTCTAGTCAGAGCCTCCGGCATGATAA
TGGATACAACTATTTGGATTGGTACCTGCAGAAGCCAGGGCAGTCTCCA
CAGCTCCTGATCTATTTGGGTTCTAAGCGGGCCTCCGGGGTCCCAGACA
GGTTCAGTGGCAGTGGATCAGGCACAGACTTTACACTGAAAATCAGTAG
AGTGGAGGCTGAGGATGTTGGGGTTTATTACTGCATGCAAACTCTACAA
ACTCTGATGTTCACTTTCGGCGGAGGGACCAAGGTGGAGATCAAAC.

In particular embodiments, a binding domain that binds HMPV includes the 7F8 antibody.

In particular embodiments, the 7F8 antibody includes a variable heavy chain including a CDRH1 including GGSISSY (SEQ ID NO: 94), a CDRH2 including YYSGS (SEQ ID NO: 95), and a CDRH3 including DYYQLPMDV (SEQ ID NO: 96), and a variable light chain including a CDRL1 including SGSSSNIGSNYVY (SEQ ID NO: 97); a CDRL2 including RNNQRPS (SEQ ID NO: 98), and a CDRL3 including AAWDDSLSGRV (SEQ ID NO: 99).

In particular embodiments, the 7F8 antibody includes a variable heavy chain including the sequence:

(SEQ ID NO: 74)
QVQLQESGPGLVKPSETLSLTCTVSGGSMSNYYWNWIRQPPGKGLEWIG
YINYSGVTRYNPSLSSRVTISVDRSKNQFSLKLSSVTAADRAVYYCARS
RSCSSDNCYMFWDYYGMDVWGQGTTVTVSS,

and a variable light chain including the sequence:

(SEQ ID NO: 75)
QSVLTQPPSASGTPGQRVTISCSGSTSNIGSNTVNWYKQLPGTAPKLLI
YSNDQRPSGVPDRFSGSKSGTSASLAISGLQSEDEADYYCAVWDDSLDG
VFGGGTKLTVL.

In particular embodiments, the 7F8 antibody includes a variable heavy chain encoded by the sequence:

(SEQ ID NO: 76)
CAGGTGCAGCTGCAGGAGTCGGGCCCAGGACTGGTGAAGCCTTCGGAGA
CCCTGTCCCTCACCTGCACTGTCTCTGGTGGCTCCATGAGCAATTACTA
CTGGAATTGGATCCGGCAGCCCCCAGGGAAGGGTCTGGAGTGGATTGGG
TATATCAACTACAGTGGGGTCACCAGATACAACCCCTCCCTCAGCAGTC
GAGTCACCATTTCAGTGGACAGGTCGAAGAACCAGTTCTCGCTGAAGTT
GAGCTCTGTGACCGCTGCGGACAGGGCCGTCTATTACTGTGCGAGATCT
CGATCTTGCAGTAGTGACAACTGCTATATGTTTTGGGACTACTACGGTA
TGGACGTCTGGGGCCAGGGGACCACGGTCACCGTCTCCTCAG,

and a variable light chain sequence encoded by the sequence:

(SEQ ID NO: 77)
CAGTCTGTGCTGACGCAGCCACCCTCAGCGTCTGGGACCCCCGGGCAGA
GGGTCACCATCTCTTGTTCTGGAAGCACCTCCAACATCGGAAGTAATAC
TGTAAACTGGTATAAGCAGCTCCCAGGAACGGCTCCCAAACTCCTCATA
TATAGTAATGATCAGCGGCCCTCAGGGGTCCCTGACCGATTCTCTGGCT
CCAAGTCTGGCACCTCAGCCTCCCTGGCCATCAGTGGGCTCCAGTCTGA
AGATGAGGCTGATTATTACTGTGCAGTCTGGGATGACAGCCTGGATGGG
GTGTTCGGCGGAGGGACCAAGCTGACCGTCCTAG.

The carboxy-terminal portion of each chain of a naturally occurring antibody defines a constant region, which can be responsible for effector function particularly in the heavy chain (the Fc). Examples of effector functions include: C1q binding and complement dependent cytotoxicity (CDC); antibody-dependent cell-mediated cytotoxicity (ADCC); phagocytosis; down regulation of cell surface receptors (e.g., B-cell receptors); and B-cell activation.

Human light chains are classified as kappa (IgK) and lambda (Igλ) light chains. In particular embodiments, a human IgK Fc region includes the sequence: RTVAAPSVFIFPPSDEQLKSGTASVVCLLNNFYPREAKVQWKVDNALQSGNSQESVTEQDSKD STYSLSSTLTLSKADYEKHKVYACEVTHQGLSSPVTKSFNRGEC (SEQ ID NO: 29). In particular embodiments, a human Igλ Fc region includes the sequence:

(SEQ ID NO: 30)
SVSVSPGQTARITCSGDALPKKYAYWYQQKSGQAPVLVIYEDNRRPSGI
PERFSGSSSRTLATLTISGAQVEDEADYYCYSTDSSGNHVVFGGGTKLT
VLRQPKAAPSVTSVPT.

Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

In particular embodiments, a human IgG1 Fc region includes the sequence:

(SEQ ID NO: 31)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In particular embodiments, a human IgG1 Fc region includes the sequence:

(SEQ ID NO: 32)
THTCPPCPAPEFFGGPSVFFFPPKPKDTFMISRTPEVTCVVVDVSHEDP
EVKFNWYVDGVEVHNAKTKPREEQYNSTYRVVSVETVFHQDWENGKEYK
CKVSNKAFPVPIEKTISKAKGQPREPQVYTLPPSREEMTKNQVSLTCLV
KGFYPSDIAVEWESNGQPENNYKTTPPVLDSDGPFFLYSKLTVDKSRWQ
QGNVFSCSVMHEALHNHYTQKSLSLSPGK.

In particular embodiments, a human IgG2 Fc region includes the amino acid sequence:

(SEQ ID NO: 33)
PAPPVAGPSVFLFPPKPKDTLMISRTPEVTCVVVDVSHEDPEVQFNWYV
DGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCS
VMHEALHNHYTQKSLSLSPGK.

In particular embodiments, a human IgG3 Fc region includes the amino acid sequence:

(SEQ ID NO: 34)
PAPELLGGPSVFLFPPKPKDTLMISRTPEVTCVVVDVSHEDPEVQFKWY
VDGVEVHNAKTKPREEQFNSTFRVVSVLTVLHQDWLNGKEYKCKVSNKA
LPAPIEKTISKTKGQPREPQVYTLPPSREEMTKNQVSLTCLVKGFYPSD
IAVEWESSGQPENNYNTTPPMLDSDGSFFLYSKLTVDKSRWQQGNIFSC
SVMHEALHNRFTQKSLSLSPGK.

In particular embodiments, a human IgG4 Fc region includes the amino acid sequence:

(SEQ ID NO: 35)
PAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKG
LPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSC
SVMHEALHNHYTQKSLSLSLGK.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989)).

Antibodies bind epitopes on antigens. The term antigen refers to a molecule or a portion of a molecule capable of being bound by an antibody. An epitope is a region of an antigen that is bound by the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics. When the antigen is a protein or peptide, the epitope includes specific amino acids within that protein or peptide that contact the variable region of an antibody.

Unless otherwise indicated, the term “antibody” includes (in addition to antibodies having two full-length heavy chains and two full-length light chains as described above) variants, derivatives, and fragments thereof, examples of which are described below. Furthermore, unless explicitly excluded, antibodies can include monoclonal antibodies, human antibodies, bispecific antibodies, trispecific antibodies, tetraspecific antibodies, multi-specific antibodies, polyclonal antibodies, linear antibodies, minibodies, domain antibodies, synthetic antibodies, chimeric antibodies, antibody fusions, and fragments thereof, respectively. In particular embodiments, antibodies can include oligomers or multiplexed versions of antibodies.

A monoclonal antibody refers to an antibody obtained from a population of substantially homogeneous antibodies, i.e., the individual antibodies including the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method. For example, monoclonal antibodies can be made by a variety of techniques, including the hybridoma method, recombinant DNA methods, phage-display methods, and methods utilizing transgenic animals containing all or part of the human immunoglobulin loci.

A “human antibody” is one which includes an amino acid sequence which corresponds to that of an antibody produced by a human or a human cell or derived from a non-human source that utilizes human antibody repertoires or other human antibody-encoding sequences.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. The subgroup of sequences can be a subgroup as in Kabat et al., Sequences of Proteins of Immunological Interest, Fifth Edition, NIH Publication 91-3242, Bethesda Md. (1991), vols. 1-3. In particular embodiments, for the V_{L}, the subgroup is subgroup kappa I as in Kabat et al. (supra). In particular embodiments, for the V_{H}, the subgroup is subgroup Ill as in Kabat et al. (supra).

Antibodies disclosed herein can be utilized to prepare various forms of relevant binding domain molecules. For example, particular embodiments can include binding fragments of an antibody, e.g., Fv, Fab, Fab′, F(ab′)₂, and single chain Fv fragments (scFvs) or any biologically effective fragments of an immunoglobulin that bind specifically to an epitope described herein.

In particular embodiments, an antibody fragment is used. An “antibody fragment” denotes a portion of a full-length antibody that retains the ability to bind to an epitope. Antibody fragments can be made by various techniques, including proteolytic digestion of an intact antibody as well as production by recombinant host-cells (e.g., mammalian suspension cell lines, E. coli or phage), as described herein. Antibody fragments can be screened for their binding properties in the same manner as intact antibodies. Examples of antibody fragments include Fv, scFv, Fab, Fab′, Fab′-SH, F(ab′)₂; diabodies; and linear antibodies.

A single chain variable fragment (scFv) is a fusion protein of the variable regions of the heavy and light chains of immunoglobulins connected with a short linker peptide. Fv fragments include the V_{L} and V_{H} domains of a single arm of an antibody but lack the constant regions.

Although the two domains of the Fv fragment, V_{L} and V_{H}, are coded by separate genes, they can be joined, using, for example, recombinant methods, by a synthetic linker that enables them to be made as a single protein chain in which the V_{L} and V_{H} regions pair to form monovalent molecules (single chain Fv (scFv)). For additional information regarding Fv and scFv, see e.g., Bird, et al., Science 242:423-426, 1988; Huston, et al., Proc. Natl. Acad. Sci. USA 85:5879-5883, 1988; Plueckthun, in The Pharmacology of Monoclonal Antibodies, vol. 113, Rosenburg and Moore (eds.), Springer-Verlag, New York), (1994) 269-315; WO 1993/16185; U.S. Pat. Nos. 5,571,894; and 5,587,458.

Linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies. Linker sequences of scFv are commonly Gly-Ser linkers, described in more detail elsewhere herein.

Additional examples of antibody-based binding domain formats include scFv-based grababodies and soluble VH domain antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

A Fab fragment is a monovalent antibody fragment including V_{L}, V_{H}, CL and CH1 domains. A F(ab′)₂ fragment is a bivalent fragment including two Fab fragments linked by a disulfide bridge at the hinge region. For discussion of Fab and F(ab′)₂ fragments having increased in vivo half-life, see U.S. Pat. No. 5,869,046. Diabodies include two epitope-binding sites that may be bivalent. See, for example, EP 0404097; WO1993/01161; and Holliger, et al., Proc. Natl. Acad. Sci. USA 90:6444-6448, 1993. Dual affinity retargeting antibodies (DART™; based on the diabody format but featuring a C-terminal disulfide bridge for additional stabilization (Moore et al., Blood 117:4542-51, 2011)) can also be used. Antibody fragments can also include isolated CDRs. For a review of antibody fragments, see Hudson, et al., Nat. Med. 9:129-134, 2003.

In particular embodiments, one or more amino acid modifications may be introduced into the Fc region of an antibody, thereby generating an Fc region variant. The Fc region variant may include a human Fc region sequence (e.g., a human IgG1, IgG2, IgG3 or IgG4 Fc region) including an amino acid modification (e.g., a substitution) at one or more amino acid positions. Numerous Fc modifications are known in the art, and a representative sampling of such possible modifications are described herein.

In particular embodiments, variants (including Fc variants) have been modified from a reference sequence to produce an administration benefit. Exemplary administration benefits can include (1) reduced susceptibility to proteolysis, (2) reduced susceptibility to oxidation, (3) altered binding affinity for forming protein complexes, (4) altered binding affinities, (5) reduced immunogenicity; and/or (6) extended half-life. While the disclosure below describes these modifications in terms of their application to antibodies, when applicable to another particular HMPV binding domain format (e.g., bispecific antibodies), the modifications can also be applied to these other formats.

In particular embodiments the antibodies can be mutated to increase their affinity for Fc receptors. Exemplary mutations that increase the affinity for Fc receptors include: G236A/S239D/A330L/1332E (GASDALIE). Smith et al., Proceedings of the National Academy of Sciences of the United States of America, 109(16), 6181-6186, 2012. In particular embodiments, an antibody variant includes an Fc region with one or more amino acid substitutions which improve ADCC, e.g., substitutions at positions 298, 333, and/or 334 of the Fc region (EU numbering of residues). In particular embodiments, alterations are made in the Fc region that result in altered C1q binding and/or Complement Dependent Cytotoxicity (CDC), e.g., as described in U.S. Pat. No. 6,194,551, WO 99/51642, and Idusogie et al., J. Immunol. 164: 4178-4184, 2000.

In particular embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further below. In particular embodiments, residue 5400 (EU numbering) of the heavy chain Fc region is selected. Cysteine engineered antibodies may be generated as described, e.g., in U.S. Pat. No. 7,521,541.

Antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at position 297 in the Fc region (Eu numbering of Fc region residues); however, Asn297 may also be located ±3 amino acids upstream or downstream of position 297, i.e., between positions 294 and 300, due to minor sequence variations in antibodies. Such fucosylation variants may have improved ADCC function. See, e.g., WO2000/61739; WO 2001/29246; WO2002/031140; US2002/0164328; WO2003/085119; WO2003/084570; US2003/0115614; US2003/0157108; US2004/0093621; US2004/0110704; US2004/0132140; US2004/0110282; US2004/0109865; WO2005/035586; WO2005/035778; WO2005/053742; Okazaki et al. J. Mol. Biol. 336:1239-1249 (2004); and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545, 1986, and knockout cell lines, such as alpha-1,6-fucosyltransferase gene, FUT8, knockout CHO cells (see, e.g., Yamane-Ohnuki et al., Biotech. Bioeng. 87: 614, 2004; Kanda et al., Biotechnol. Bioeng., 94(4):680-688, 2006; and WO2003/085107).

In particular embodiments, modified antibodies include those wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid, a farnesylated amino acid, an acetylated amino acid, a biotinylated amino acid, an amino acid conjugated to a lipid moiety, or an amino acid conjugated to an organic derivatizing agent. Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Modifications also include nitrited constructs.

In particular embodiments, variants include glycosylation variants wherein the number and/or type of glycosylation site has been altered compared to the amino acid sequences of a reference sequence. In particular embodiments, glycosylation variants include a greater or a lesser number of N-linked glycosylation sites than the reference sequence. An N-linked glycosylation site is characterized by the sequence: Asn-X-Ser or Asn-X-Thr, wherein the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided is a rearrangement of N-linked carbohydrate chains wherein one or more N-linked glycosylation sites (e.g., those that are naturally occurring) are eliminated and one or more new N-linked sites are created. Additional antibody variants include cysteine variants wherein one or more cysteine residues are deleted from or substituted for another amino acid (e.g., serine) as compared to the reference sequence. These cysteine variants can be useful when antibodies must be refolded into a biologically active conformation such as after the isolation of insoluble inclusion bodies. These cysteine variants generally have fewer cysteine residues than the reference sequence, and typically have an even number to minimize interactions resulting from unpaired cysteines.

PEGylation particularly is a process by which polyethylene glycol (PEG) polymer chains are covalently conjugated to other molecules such as proteins. Several methods of PEGylating proteins have been reported in the literature. For example, N-hydroxy succinimide (NHS)-PEG was used to PEGylate the free amine groups of lysine residues and N-terminus of proteins; PEGs bearing aldehyde groups have been used to PEGylate the amino-termini of proteins in the presence of a reducing reagent; PEGs with maleimide functional groups have been used for selectively PEGylating the free thiol groups of cysteine residues in proteins; and site-specific PEGylation of acetyl-phenylalanine residues can be performed.

Covalent attachment of proteins to PEG has proven to be a useful method to increase the half-lives of proteins in the body (Abuchowski, A. et al., Cancer Biochem. Biophys., 1984, 7:175-186; Hershfield, M. S. et al., N. Engl. J. Medicine, 1987, 316:589-596; and Meyers, F. J. et al., Clin. Pharmacol. Ther., 49:307-313, 1991). The attachment of PEG to proteins not only protects the molecules against enzymatic degradation, but also reduces their clearance rate from the body. The size of PEG attached to a protein has significant impact on the half-life of the protein. The ability of PEGylation to decrease clearance is generally not a function of how many PEG groups are attached to the protein, but the overall molecular weight of the altered protein. Usually the larger the PEG is, the longer the in vivo half-life of the attached protein. In addition, PEGylation can also decrease protein aggregation (Suzuki et al., Biochem. Bioph. Acta 788:248, 1984), alter protein immunogenicity (Abuchowski et al., J. Biol. Chem. 252: 3582, 1977), and increase protein solubility as described, for example, in PCT Publication No. WO 92/16221).

Several sizes of PEGs are commercially available (Nektar Advanced PEGylation Catalog 2005-2006; and NOF DDS Catalogue Ver 7.1), which are suitable for producing proteins with targeted circulating half-lives. A variety of active PEGs have been used including mPEG succinimidyl succinate, mPEG succinimidyl carbonate, and PEG aldehydes, such as mPEG-propionaldehyde.

In particular embodiments, the antibody can be fused or coupled to an Fc polypeptide that includes amino acid alterations that extend the in vivo half-life of an antibody that contains the altered Fc polypeptide as compared to the half-life of a similar antibody containing the same Fc polypeptide without the amino acid alterations. In particular embodiments, Fc polypeptide amino acid alterations can include M252Y, S254T, T256E, M428L, and/or N434S and can be used together, separately or in any combination. For example, M428L/N434S is a pair of mutations that increase the half-life of antibodies in serum, as described in Zalevsky et al., Nature Biotechnology 28, 157-159, 2010. Other alterations that can be helpful are described in U.S. Pat. Nos. 7,083,784, 7,670,600, US Publication No. 2010/0234575, PCT/US2012/070146, and Zwolak, Scientific Reports 7: 15521, 2017. In particular embodiments, any substitution at one of the following amino acid positions in an Fc polypeptide can be considered an Fc alteration that extends half-life: 250, 251, 252, 259, 307, 308, 332, 378, 380, 428, 430, 434, 436. Each of these alterations or combinations of these alterations can be used to extend the half-life of a bispecific antibody as described herein.

### (ii) Multi-Domain Binding Molecules

Multi-domain binding molecules include at least two binding domains, wherein at least one binding domain includes an HMPV binding domain disclosed herein. In particular embodiments, a multi-domain binding molecule includes at least one, at least two, at least, three, at least four binding domains that bind an epitope on HMPV. In particular embodiments, all of the binding domains of a multi-domain binding molecule bind HMPV. In particular embodiments, multi-domain binding molecules include bispecific antibodies, trispecific antibodies, and so on.

Bispecific antibodies can be prepared as full-length antibodies or antibody fragments (for example, F(ab′)₂ bispecific antibodies). For example, WO 1996/016673 describes a bispecific anti-ErbB2/anti-Fc gamma RIII antibody; U.S. Pat. No. 5,837,234 describes a bispecific anti-ErbB2/anti-Fc gamma RI antibody; WO 1998/002463 describes a bispecific anti-ErbB2/Fc alpha antibody; and U.S. Pat. No. 5,821,337 describes a bispecific anti-ErbB2/anti-CD3 antibody. In particular embodiments, a bispecific antibody can be in the form of a Bispecific T-cell Engaging (BiTE®) antibody.

Some additional exemplary bispecific antibodies have two heavy chains (each having three heavy chain CDRs, followed by (N-terminal to C-terminal) a CH1 domain, a hinge, a CH2 domain, and a CH3 domain), and two immunoglobulin light chains that confer antigen-binding specificity through association with each heavy chain. However, as indicated, additional architectures are envisioned, including bi-specific antibodies in which the light chain(s) associate with each heavy chain but do not (or minimally) contribute to antigen-binding specificity, or that can bind one or more of the epitopes bound by the heavy chain antigen-binding regions, or that can associate with each heavy chain and enable binding of one or both of the heavy chains to one or both epitopes.

scFv dimers or diabodies may be used, rather than whole antibodies. Diabodies and scFv can be constructed without an Fc region, using only variable domains (usually including the variable domain components from both light and heavy chains of the source antibody), potentially reducing the effects of anti-idiotypic reaction. Other forms of bispecific antibodies include the single chain “Janusins” described in Traunecker et al. (Embo Journal, 10, 3655-3659, 1991).

Bispecific antibodies with extended half-lives are described in, for example, U.S. Pat. No. 8,921,528 and US Patent Publication No. 2014/0308285.

Methods for making bispecific antibodies are known in the art. For example, traditional production of full-length bispecific antibodies is based on the co-expression of two immunoglobulin heavy chain-light chain pairs, where the two chains have different specificities (see, for example, Millstein et al. Nature 305:37-39, 1983). Similar procedures are disclosed in, for example, WO 1993/008829, Traunecker et al., EMBO J. 10:3655-3659, 1991 and Holliger & Winter, Current Opinion Biotechnol. 4, 446-449 (1993).

In particular embodiments, bispecific antibodies can be prepared using chemical linkage. For example, Brennan et al. (Science 229: 81, 1985) describes a procedure wherein intact antibodies are proteolytically cleaved to generate F(ab′)2 fragments. These fragments are reduced in the presence of the dithiol complexing agent, sodium arsenite, to stabilize vicinal dithiols and prevent intermolecular disulfide formation. The Fab′ fragments generated then are converted to thionitrobenzoate (TNB) derivatives. One of the Fab′-TNB derivatives then is reconverted to the Fab′-thiol by reduction with mercaptoethylamine and is mixed with an equimolar amount of the other Fab′-TNB derivative to form the bispecific antibody.

In particular embodiments, bispecific antibodies can be prepared using knobs-into holes techniques. Knobs-into-holes refers to forcing the pairing of two different antibody heavy chains by introducing mutations into the CH3 domains to modify the contact interface. On one chain bulky amino acids are replaced by amino acids with short side chains to create a ‘hole’. Conversely, amino acids with large side chains were introduced into the other CH3 domain, to create a ‘knob’. By coexpressing these two heavy chains (and two identical light chains, which have to be appropriate for both heavy chains), high yields of heterodimer formation (‘knob-hole’) versus homodimer formation (‘hole-hole’ or ‘knob-knob’) is observed (Ridgway, J. B., Protein Eng. 9 (1996) 617-621; and WO 96/027011).

In particular embodiments, the ‘knob’ and/or the ‘hole’ may exist in the original polypeptide or may be introduced synthetically (e.g., by altering nucleic acid encoding the polypeptide). To synthetically introduce a knob and/or hole, the nucleic acid encoding the original amino acid residue (or other non-amino acid groups such as, for example carbohydrate groups) in the interface of the polypeptide is replaced with DNA encoding at least one import amino acid residue, wherein the interface refers to amino acid residues in contact between a first heavy chain constant region and one or more amino acid residues (or other non-amino acid groups) in a second heavy chain constant region. The preferred import residues for the formation of a hole are amino acids with smaller side chain volumes than the original amino acid residue such as alanine (A), serine (S), threonine (T), valine (V), or glycine (G). The preferred import residues for the formation of a knob are amino acids with larger side chain volumes than the original amino acid residue such as tyrosine (Y), arginine (R), phenylalanine (F), or tryptophan (W). The percentage of heterodimer can be increased by remodeling the interaction surfaces of the two CH3 domains using a phage display approach and the introduction of a disulfide bridge to stabilize the heterodimers (Merchant A. M, et al., Nature Biotech 16 (1998) 677-681; Atwell, S., et al., J. Mol. Biol. 270 (1997) 26-35).

Two or more antibodies or fragment thereof can be linked through a linker to form a multi-domain binding molecule. Examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target.

Commonly used flexible linkers include linker sequence with the amino acids glycine and serine (Gly-Ser linkers). In particular embodiments, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10). Particular examples include (Gly₄Ser)ₙ (SEQ ID NO: 36), (Gly₃Ser)ₙ(Gly₄Ser)ₙ (SEQ ID NO: 37), (Gly₃Ser)ₙ(Gly₂Ser)ₙ (SEQ ID NO: 38), and (Gly₃Ser)ₙ(Gly₄Ser)₁ (SEQ ID NO: 39). In particular embodiments, the linker is (Gly₄Ser)₄ (SEQ ID NO: 40), (Gly₄Ser)₃ (SEQ ID NO: 41), (Gly₄Ser)₂ (SEQ ID NO: 42), (Gly₄Ser)₁ (SEQ ID NO: 43), (Gly₃Ser)₂ (SEQ ID NO: 44), (Gly₃Ser)₁ (SEQ ID NO: 45), (Gly₂Ser)₂ (SEQ ID NO: 46) or (Gly₂Ser)₁, GGSGGGSGGSG (SEQ ID NO: 47), GGSGGGSGSG (SEQ ID NO: 48), or GGSGGGSG (SEQ ID NO: 49).

Linkers that include one or more antibody hinge regions and/or immunoglobulin heavy chain constant regions, such as CH3 alone or a CH2CH3 sequence can also be used. Additional examples of linkers can be found in Chen et al., Adv Drug Deliv Rev. 2013 Oct. 15; 65(10): 1357-1369. Linkers can be flexible, rigid, or semi-rigid, depending on the desired functional domain presentation to a target.

In some situations, flexible linkers may be incapable of maintaining a distance or positioning of binding domains needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

In particular embodiments, binding fragments disclosed herein can be used to create bi-tri-, quad- (or more) specific antibody constructs that bind HMPV and a secondary virus. In particular embodiments, the secondary virus is selected from respiratory syncytial virus (RSV), adenovirus, a boca virus, a coronavirus (e.g., severe acute respiratory syndrome (SARS)-coronoavirus (CoV), Middle Ease respiratory syndrome CoV, coronavirus 229E, coronavirus OC43, coronavirus NL63, coronavirus HKU1, coronavirus NL, coronavirus NH), an enterovirus, an influenza virus (groups A and B), a metapneumovirus, a parainfluenza virus (human parainfluenza virus (HPIV)), and/or a rhinovirus (human rhinovirus (HRV A-HRV C)).

T-cell activation can be mediated by two distinct signals: those that initiate antigen-dependent primary activation and provide a T-cell receptor like signal (primary cytoplasmic signaling sequences) and those that act in an antigen independent manner to provide a secondary or co-stimulatory signal (secondary cytoplasmic signaling sequences). I-AMS disclosed herein can target any T-cell activating epitope that upon binding induces T-cell activation. Examples of such T-cell activating epitopes are on T-cell markers including CD2, CD3, CD7, CD27, CD28, CD30, CD40, CD83, 4-1BB (CD 137), OX40, lymphocyte function-associated antigen-1 (LFA-1), LIGHT, NKG2C, and B7-H3. Binding domains that bind T-cell markers are known in the art.

In particular embodiments macrophages are targeted for localized activation by I-AMS. Macrophages are a type of leukocyte (or white blood cell) that can engulf and digest cells, cellular debris, and/or foreign substances in a process known as phagocytosis.

The I-AMS can be designed to bind to a protein expressed on the surface of macrophages. Examples of activating proteins expressed on the surface of macrophages (and their precursors, monocytes) include CD11b, CD11c, CD64, CD68, CD119, CD163, CD206, CD209, F4/80, IFGR2 Toll-like receptors (TLRs) 1-9, IL-4Rα, and MARCO. Commercially available antibodies that bind to proteins expressed on the surface of macrophages include M1/70, which binds and activates CD11b (available from BioLegend®); KP1, which binds and activates CD68 (available from ABCAM®, Cambridge, United Kingdom); and ab87099, which binds and activates CD163 (available from ABCAM®).

In particular embodiments, I-AMS can target a pathogen recognition receptor (PRR). PRRs are proteins or protein complexes that recognize a danger signal and activate and/or enhance the innate immune response. Examples of PRRs include the TLR4/MD-2 complex, which recognizes gram negative bacteria; Dectin-1 and Dectin-2, which recognize mannose moieties on fungus and other pathogens; TLR2/TLR6 or TLR2/TLR1 heterodimers, which recognize gram positive bacteria; TLR5, which recognizes flagellin; and TLR9 (CD289), which recognizes CpG motifs in DNA. In particular embodiments, I-AMS can bind and activate TLR4/MD-2, Dectin-1, Dectin-2, TRL2/TLR6, TLR2/TLR1, TLR5, and/or TLR9.

In particular embodiments, I-AMS can target the complement system. The complement system refers to an immune pathway that is induced by antigen-bound antibodies and involves signaling of complement proteins, resulting in immune recognition and clearance of the antibody-coated antigens.

Binding domains of I-AMS and other engineered formats described herein may be joined through a linker. A linker is an amino acid sequence which can provide flexibility and room for conformational movement between the binding domains of a I-AM. Any appropriate linker may be used.

Examples of linkers can be found in Chen et al. (Adv Drug Deliv Rev. 2013 Oct. 15; 65(10): 1357-1369) and described elsewhere herein. Linkers can be flexible, rigid, or semi-rigid, depending on the desired functional domain presentation to a target.

Cytolytic properties of I-AMS molecules can be confirmed in comparative in vitro assays. Briefly, for cell line experiments, target cells can be incubated in 96-well round bottom plates at 5-10,000 cells/well containing increasing concentrations of the various I-AMS antibodies with/without healthy donor T-cells (used at an E:T cell ratio of 1:1 and 3:1). After 48 hours, cell numbers and drug-induced cytotoxicity, using 4′,6-diamidino-2-phenylindole (DAPI) to detect non-viable cells, can be determined by flow cytometry. In experiments where healthy donor T-cells are added, cells can be identified by forward/side scatter properties and negativity for CellVue Burgundy dye. Experiments can include technical duplicates.

In particular embodiments including I-AMS constructs, T-cell activating epitope binding domains include one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the V_{α}, V_{β}, C_{α}, or C_{β} of a known TCR. An insertion, deletion or substitution may be anywhere in a V_{α}, V_{β}, C_{α}, or C_{β} region, including at the amino- or carboxy-terminus or both ends of these regions, provided that each CDR includes zero changes or at most one, two, or three changes and provided a binding domain including a modified V_{α}, V_{β}, C_{α}, or C_{α} region can still specifically bind its target with an affinity similar to wild type.

Tri-specific antibodies are artificial proteins that simultaneously bind to three different types of antigens, wherein at least one of the antigens is HMPV. Tri-specific antibodies are described in, for example, WO2016/105450, WO 2010/028796; WO 2009/007124; WO 2002/083738; US 2002/0051780; and WO 2000/018806.

In some embodiments, a multi-domain binding molecule includes a basic immunoglobulin structure such as an IgA domain or an IgM domain. Basic immunoglobulin structures in vertebrate systems are described above and are well understood. (See, e.g., Harlow et al., Antibodies: A Laboratory Manual, Cold Spring Harbor Laboratory Press, 2nd ed. 1988).

In particular embodiments, multi-domain binding molecules are multimers of an antibody disclosed herein. Multimerization strategies include formation of a fusion protein using protein linkers or use of IgA or IgM constant regions as a multimerization scaffold. In certain aspects, multimerization is achieved by linking antibodies or binding domains of antibodies in a fusion protein with protein linkers. Fusion proteins include different protein domains linked to each other directly or through intervening linker segments such that the function of each included domain is retained.

Multimerized antibodies and antibody-like molecules such as IgA and IgM antibodies have emerged as promising drug candidates in the fields of, e.g., immuno-oncology and infectious diseases allowing for improved specificity, improved avidity, and the ability to bind to multiple binding targets.

Particular embodiments include using IgA and IgM constant region domains to allow the binding portion of molecules provided herein to multimerize.

Immunoglobulin A (IgA), as the major class of antibody present in the mucosal secretions of most mammals, represents a key first line of defense against invasion by inhaled and ingested pathogens. IgA is also found at significant concentrations in the serum of many species, where it functions as a second line of defense mediating elimination of pathogens that have breached the mucosal surface. Receptors specific for the Fc region of IgA, FcaR, are key mediators of IgA effector function. Native IgA is a tetrameric protein including two identical light chains (K or λ) and two identical heavy chains. IgA, similarly to IgG, contains three constant domains (CA1-CA3), with a hinge region between the CA1 and CA2 domains. The main difference between IgA1 and IgA2 resides in the hinge region that lies between the two Fab arms and the Fc region. IgA1 has an extended hinge region due to the insertion of a duplicated stretch of amino acids, which is absent in IgA2. Both forms of IgA have the capacity to form dimers, in which two monomer units, are arranged in an end-to-end configuration stabilized by disulfide bridges and incorporation of a J-chain. J-chains are also part of IgM pentamers and are discussed in more detail below.

Both IgA and IgM (discussed further below in relation to pentamers and hexamers) possess an 18-amino acid extension in the C terminus called the “tail-piece” (tp). The IgA and IgM tp is highly conserved among various animal species. The conserved penultimate cysteine residue in the IgA and IgM tp has been demonstrated to be involved in multimerization by forming a disulfide bond between heavy chains to permit formation of a multimer. Both tp contain an N-linked carbohydrate addition site, the presence of which is required for dimer formation in IgA and J-chain incorporation and pentamer formation in IgM. However, the structure and composition of the N-linked carbohydrates in the tp differ, suggesting differences in the accessibility of the glycans to processing by glycosyltransferases. Particularly, the IgA (atp) and IgM (μtp) tp differ at seven amino acid positions.

The human IgA1 constant region typically includes the amino acid sequence:

(SEQ ID NO: 50)
ASPTSPKVFPLSLCSTQPDGNVVIACLVQGFFPQEPLSVTWSESGQGVT
ARNFPPSQDASGDLYTTSSQLTLPATQCLAGKSVTCHVKHYTNPSQDVT
VPCPVPSTPPTPSPSTPPTPSPSCCHPRLSLHRPALEDLLLGSEANLTC
TLTGLRDASGVTFTWTPSSGKSAVQGPPERDLCGCYSVSSVLPGCAEPW
NHGKTFTCTAAYPESKTPLTATLSKSGNTFRPEVHLLPPPSEELALNEL
VTLTCLARGFSPKDVLVRWLQGSQELPREKYLTWASRQEPSQGTTTFAV
TSILRVAAEDWKKGDTFSCMVGHEALPLAFTQKTIDRLAGKPTHVNVSV
VMAEVDGTCY.

The human IgA2 constant region typically includes the amino acid sequence

(SEQ ID NO: 51)
ASPTSPKVFPLSLDSTPQDGNVVVACLVQGFFPQEPLSVTWSESGQNVT
ARNFPPSQDASGDLYTTSSQLTLPATQCPDGKSVTCHVKHYTNPSQDVT
VPCPVPPPPPCCHPRLSLHRPALEDLLLGSEANLTCTLTGLRDASGATF
TWTPSSGKSAVQGPPERDLCGCYSVSSVLPGCAQPWNHGETFTCTAAHP
ELKTPLTANITKSGNTFRPEVHLLPPPSEELALNELVTLTCLARGFSPK
DVLVRWLQGSQELPREKYLTWASRQEPSQGTTTFAVTSILRVAAEDWKK
GDTFSCMVGHEALPLAFTQKTIDRLAGKPTHVNVSVVMAEVDGTCY.

An exemplary mature secretory component includes

(SEQ ID NO: 54)
KSPIFGPEEVNSVEGNSVSITCYYPPTSVNRHTRKYWCRQGARGGCITL
ISSEGYVSSKYAGRANLTNFPENGTFVVNIAQLSQDDSGRYKCGLGINS
RGLSFDVSLEVSQGPGLLNDTKVYTVDLGRTVTINCPFKTENAQKRKSL
YKQIGLYPVLVIDSSGYVNPNYTGRIRLDIQGTGQLLFSVVINQLRLSD
AGQYLCQAGDDSNSNKKNADLQVLKPEPELVYEDLRGSVTFHCALGPEV
ANVAKFLCRQSSGENCDVVVNTLGKRAPAFEGRILLNPQDKDGSFSVVI
TGLRKEDAGRYLCGAHSDGQLQEGSPIQAWQLFVNEESTIPRSPTVVKG
VAGGSVAVLCPYNRKESKSIKYWCLWEGAQNGRCPLLVDSEGWVKAQYE
GRLSLLEEPGNGTFTVILNQLTSRDAGFYWCLTNGDTLWRTTVEIKIIE
GEPNLKVPGNVTAVLGETLKVPCHFPCKFSSYEKYWCKWNNTGCQALPS
QDEGPSKAFVNCDENSRLVSLTLNLVTRADEGWYWCGVKQGHFYGETAA
VYVAVEERKAAGSRDVSLAKADAAPDEKVLDSGFREIENKAIQDPR.

Particular embodiments include IgM immunoglobulin constant region domains that allow the binding portion of molecules provided herein to readily multimerize into pentamers or hexamers.

Particular embodiments include IgM constant regions (or variants thereof).

A “multimerizing fragment” of an IgM heavy chain constant region includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains.

Five IgM monomers form a complex with a J-chain to form a native IgM molecule. The J-chain is considered to facilitate polymerization of p chains before IgM is secreted from antibody-producing cells. Sequences for the human IGJ gene are known in the art, for example, (IGMT Accession: J00256, X86355, M25625, AJ879487). The J chain establishes the disulfide bridges between IgM antibodies to form multimeric structures such as pentamers. See, for example, Sorensen et al. International Immunology, (2000), pages 19-27. In an IgM antibody where each binding unit is bivalent, the binding molecule itself can have 10 or 12 valencies.

Exemplary multimeric binding molecules provided herein include human IgM constant regions that include the wild-type human Cμ2, Cμ3, and Cμ4-tp domains as follows:

(SEQ ID NO: 55)
VIAELPPKVSVFVPPRDGFFGNPRKSKLICQATGFSPRQIQVSWLREGK
QVGSGVTTDQVQAEAKESGPTTYKVTSTLTIKESDWLSQSMFTCRVDHR
GLTFQQNASSMCVPDQDTAIRVFAIPPSFASIFLTKSTKLTCLVTDLTT
YDSVTISWTRQNGEAVKTHTNISESHPNATFSAVGEASICEDDWNSGER
FTCTVTHTDLPSPLKQTISRPKGVALHRPDVYLLPPAREQLNLRESATI
TCLVTGFSPADVFVQWMQRGQPLSPEKYVTSAPMPEPQAPGRYFAHSIL
TVSEEEWNTGETYTCVVAHEALPNRVTERTVDKSTGKPTLYNVSLVMSD
TAGTCY.

The human IgM heavy chain constant region typically includes the amino acid sequence GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL SQSMFTCRVDHRGLTFQQNASSMCVPDQDTAIRVFAIPPSFASIFLTKSTKLTCLVTDLTTYDSV TISWTRQNGEAVKTHTNISESHPNATFSAVGEASICEDDWNSGERFTCTVTHTDLPSPLKQTIS RPKGVALHRPDVYLLPPAREQLNLRESATITCLVTGFSPADVFVQWMQRGQPLSPEKYVTSAP MPEPQAPGRYFAHSILTVSEEEWNTGETYTCVVAHEALPNRVTERTVDKSTGKPTLYNVSLVM SDTAGTCY (SEQ ID NO: 58; identical to, e.g., GenBank Accession Nos. pir∥S37768, CAA47708.1, and CAA47714.1).

In particular embodiments, an IgM heavy chain constant region includes the sequence: GSASAPTLFPLVSCENSPSDTSSVAVGCLAQDFLPDSITFSWKYKNNSDISSTRGFPSVLRGGK YAATSQVLLPSKDVMQGTDEHVVCKVQHPNGNKEKNVPLPVIAELPPKVSVFVPPRDGFFGNP RKSKLICQATGFSPRQIQVSWLREGKQVGSGVTTDQVQAEAKESGPTTYKVTSTLTIKESDWL GQSMFTCRVDHRGLTFQQNASSMCVPDQDTAIRVFAIPPSFASIFLTKSTKLTCLVTDLTTYDS VTISWTRQNGEAVKTHTNISESHPNATFSAVGEASICEDDWNSGERFTCTVTHTDLPSPLKQTI SRPKGVALHRPDVYLLPPAREQLNLRESATITCLVTGFSPADVFVQWMQRGQPLSPEKYVTSA PMPEPQAPGRYFAHSILTVSEEEWNTGETYTCVVAHEALPNRVTERTVDKSTGKPTLYNVSLV MSDTAGTCY (SEQ ID NO: 59; (UniProt ID P01871)-allele IGHM*04). This sequence differs from SEQ ID NO: 58 by one amino acid at position 191.

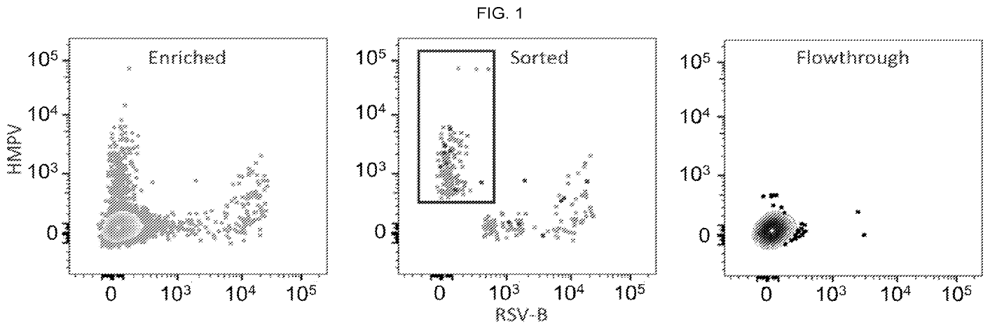

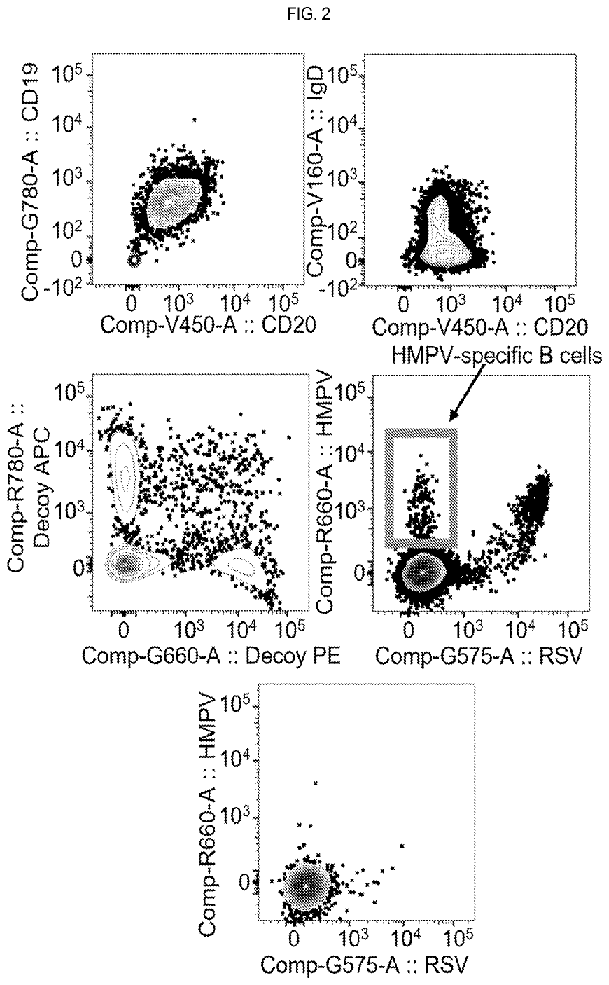

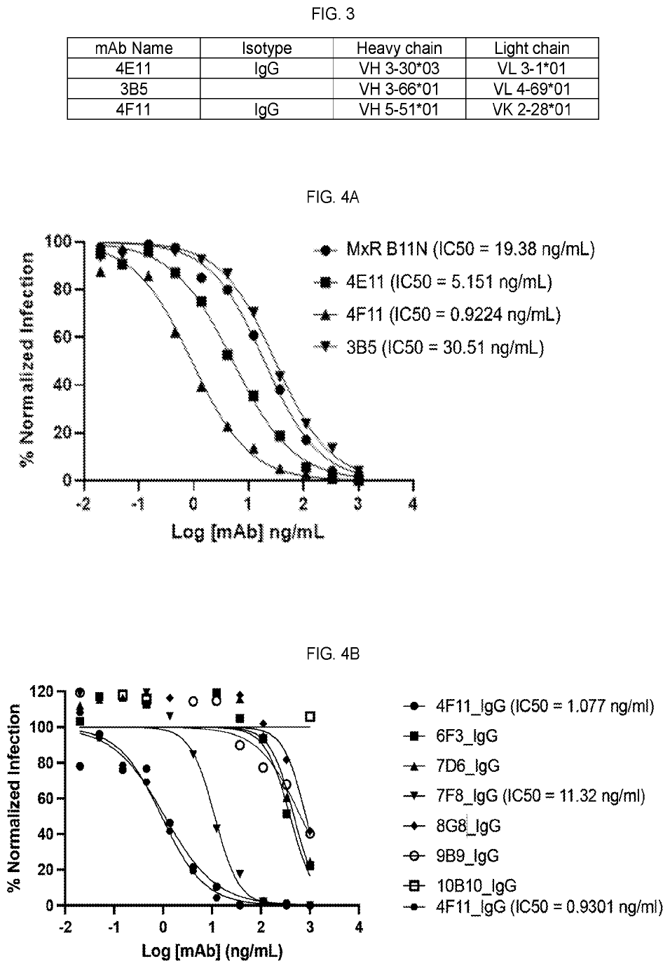

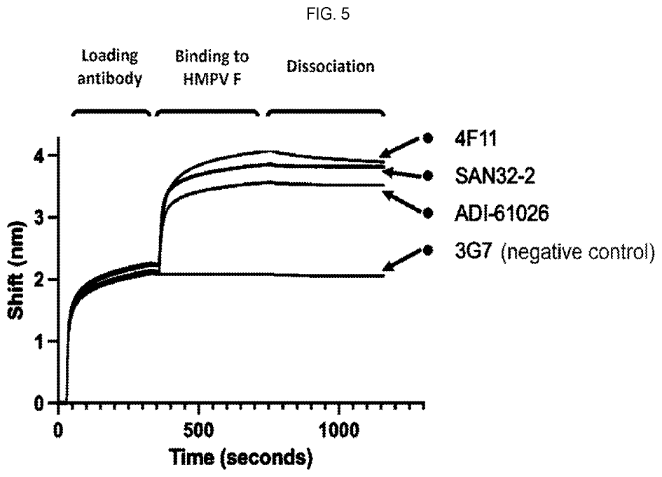

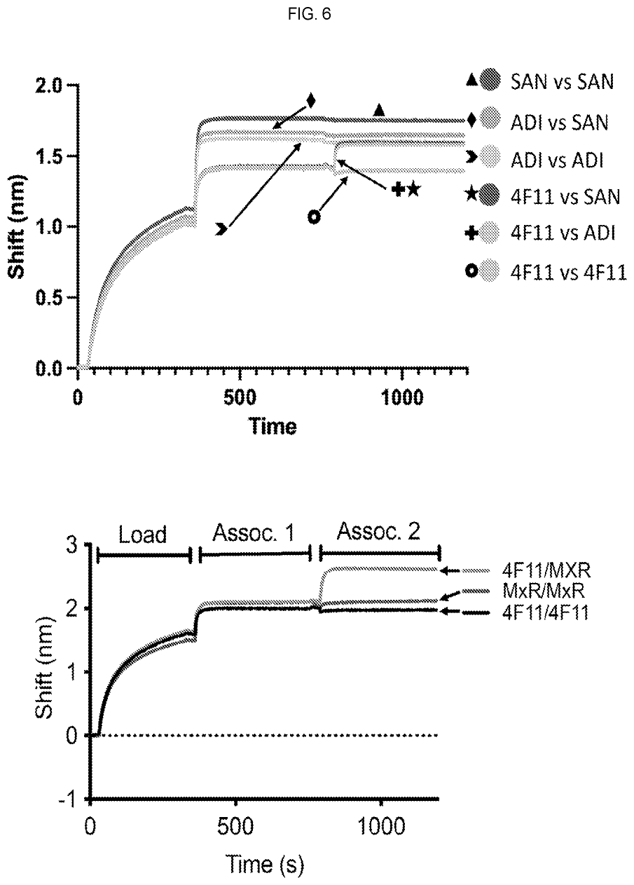

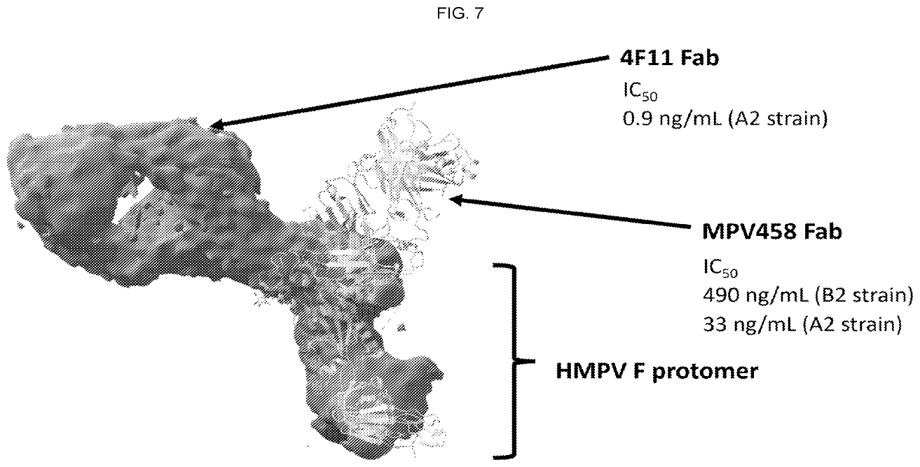

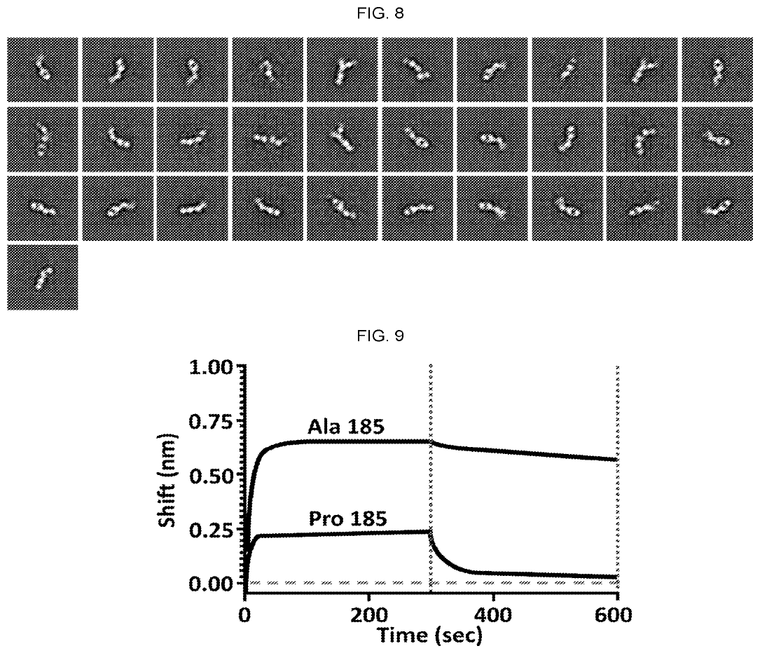

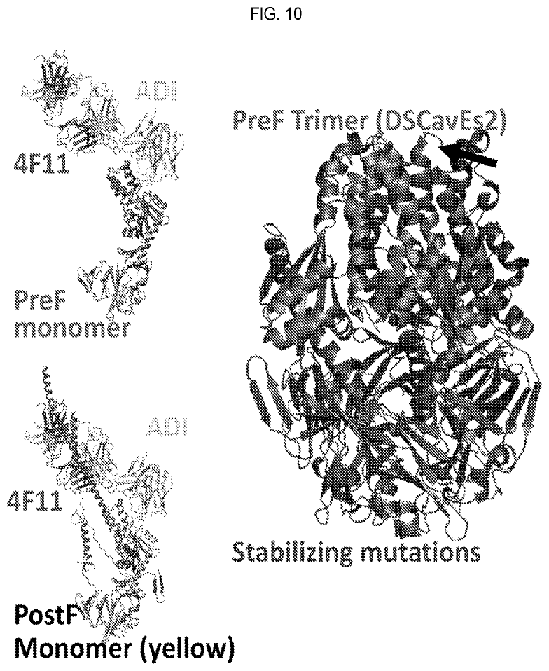

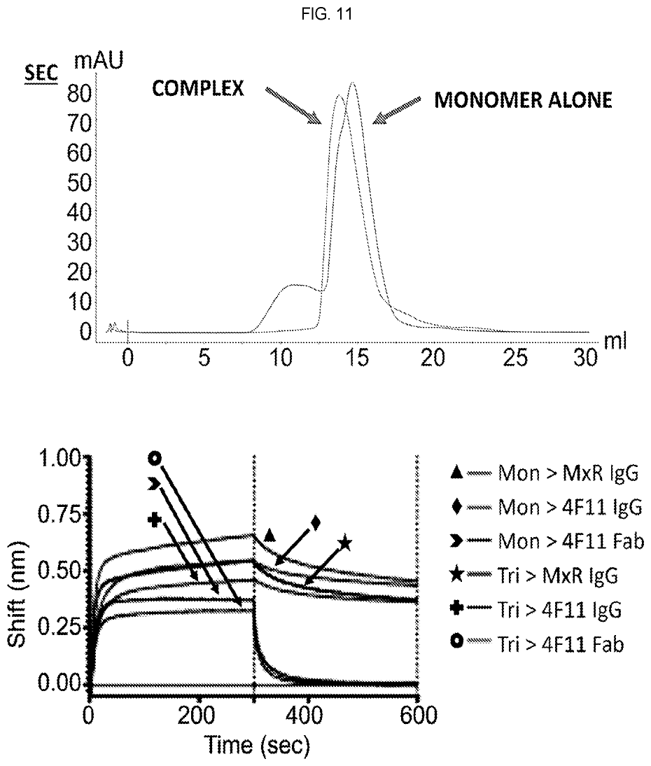

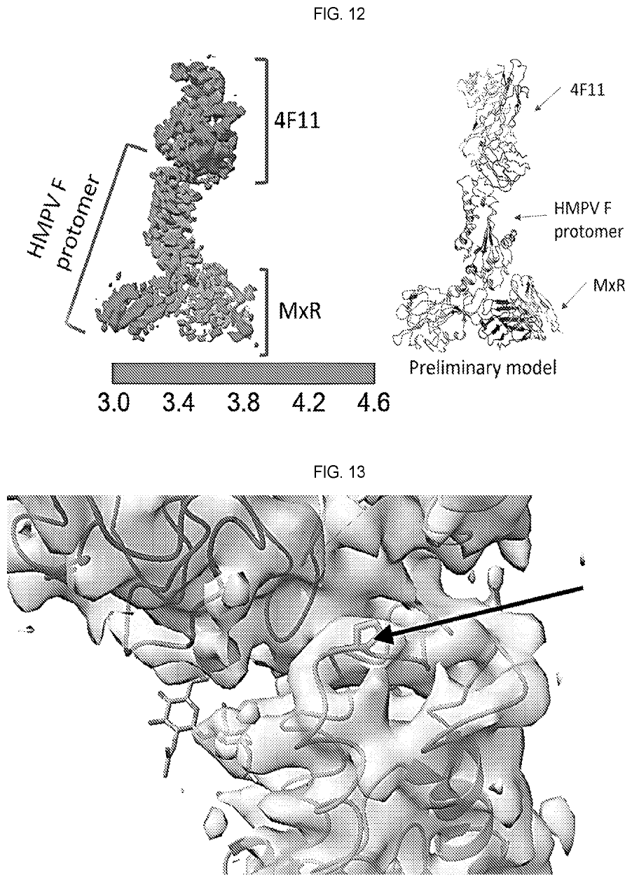

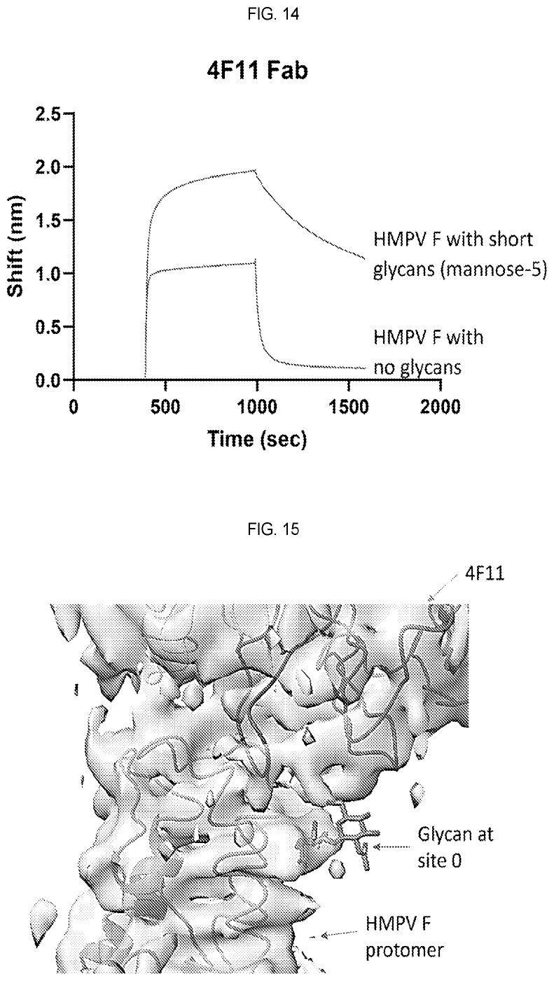

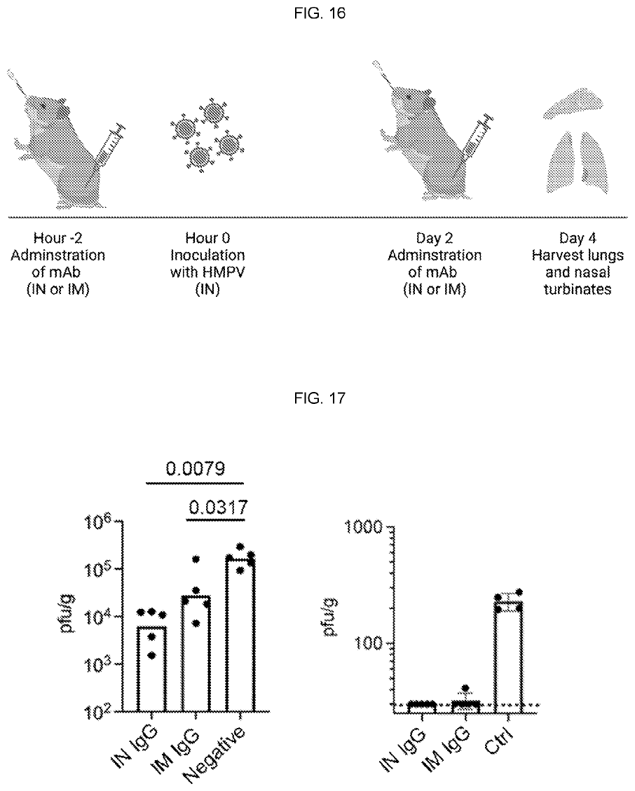

Other forms of the human IgM constant region with minor sequence variations exist, including GenBank Accession Nos. P01871.4, CAB37838.1, and pir∥MHHU. The amino acid substitutions, insertions, and/or deletions at positions corresponding to SEQ ID NO: 58 described herein can likewise be incorporated into alternate human IgM sequences, as well as into IgM constant region amino acid sequences of other species, e.g., those shown in FIG. 1 of PCT/US2019/020374.

In certain aspects, a variant human IgM constant region includes an amino acid substitution corresponding to the wild-type human IgM constant region at position P311, P313, R344, E345, S401, E402, and/or E403 of SEQ ID NO: 58. These positions correspond to the Kabat numbering system as follows: S401 of SEQ ID NO: 58 corresponds to S524 of Kabat; E402 of SEQ ID NO: 58 corresponds to E525 of Kabat; E403 of SEQ ID NO: 58 corresponds to E526 of Kabat; R344 of SEQ ID NO: 58 corresponds to R467 of Kabat; and E345 of SEQ ID NO: 58 corresponds to E468 of Kabat.

A functional fragment, derivative, and/or variant of a J-chain has at least 90% sequence identity to the reference J-chain and retains the multimerizing function of the reference J-chain.

Particular embodiments include a heterologous polypeptide (e.g., a single-domain antibody binding domain) fused to the J-chain or functional fragment thereof via a peptide linker, e.g., a peptide linker including at least 5 amino acids, but no more than 25 amino acids. In certain aspects, the peptide linker includes (GGGGS)ₙ (SEQ ID NO: 72) wherein n is 1-5.

J-chains from the following species can also be used in certain embodiments: Pan troglodytes, Pongo abelii, Callithrix jacchus, Macaca mulatta, Papio Anubis, Saimiri boliviensis, Tupaia chinensis, Tursiops truncatus, Orcinus orca, Loxodonta Africana, Leptonychotes weddellii, Ceratotherium simum, Felis catus, Canis familiaris, Ailuropoda melanoleuca, Mustela furo, Equus caballus, Cavia porcellus, Camelus ferus, Capra hircus, Chinchilla lanigera, Mesocricetus auratus, Ovis aries, Myotis lucifugus, Pantholops hodgsonii, Bos taurus, Mus musculus, Rattus norvegicus, Echinops telfairi, Oryctolagus cuniculus, Monodelphis domestica, Alligator mississippiensis, Chrysemys picta, Sarcophilus harrisii, Ornithorhynchus anatinus, Melopsittacus undulatus, Anas platyrhynchos, Gallus gallus, Meleagris gallopavo, Falco peregrinus, Zonotrichia albicollis, and Pteropus alecto.

In particular embodiments, the antibodies can multimerize by optionally including a multimerization domain. A “multimerization domain” is a domain that causes two or more proteins (monomers) to interact with each other through covalent and/or non-covalent association(s).

Multimerization domains present in proteins can result in protein interactions that form dimers, trimers, tetramers, pentamers, hexamers, heptamers, etc., depending on the number of units/monomers incorporated into the multimer.

In particular embodiments, the multimerization domain is a dimerization domain that allows binding of two complementary monomers to form a dimer. In particular embodiments, a dimerization and docking domain (DDD) can be derived from the cAMP-dependent protein kinase (PKA) regulatory subunits and can be paired with an anchoring domain (AD). The AD can be derived from a specific region found in various A-kinase anchoring proteins (AKAPs) that mediates association with the R subunits of PKA. Additional DDDs and ADs include: the 4-helix bundle type DDD (Newlon, et al. EMBO J. 2001; 20: 1651-1662; Newlon, et al. Nature Struct Biol. 1999; 3: 222-227) domains obtained from p53, DCoH (pterin 4 α carbinolamine dehydratase/dimerization cofactor of hepatocyte nuclear factor 1 α (TCF1)) and HNF-1 (hepatocyte nuclear factor 1) (Rose, et al. Nature Struct Biol. 2000; 7: 744-748). Other AD sequences of potential use may be found in US 2003/0232420A1.

In particular embodiments, complementary binding domains can dimerize. In particular embodiments, the binding domain is a transmembrane polypeptide derived from a FcεRI chain. In particular embodiments, an antibody or fragment thereof can include a part of a FcεRI α chain and another antibody or fragment thereof can include a part of an FcεRI β chain such that said FcεRI chains spontaneously dimerize together to form a dimeric antibody (e.g., bispecific antibody). In particular embodiments, an antibody or fragment thereof can include a part of a FcεRI α chain and another antibody or fragment thereof part of a FcεRI γ chain such that said FcεRI chains spontaneously trimerize together to form a trimeric antibody, and in another embodiment the multi-domain binding molecule can include a part of FcεRI α chain, a part of FcεRI βchain and a part of FcεRI γ chain such that said FcεRI chains spontaneously tetramerize together to form a tetrameric multi-domain binding molecule.

Leucine zippers are described in U.S. Pat. No. 5,932,448; SH2 and SH3 are described in Vidal et al., Biochemistry, 43:7336-44, 2004); PTB is described in Zhou et al., Nature, 378:584-592, 1995); WW is described in Sudol Prog Biochys MoL Bio, 65:113-132, 1996; PDZ is described in Kim et al., Nature, 378: 85-88, 1995 and Komau et al., Science, 269:1737-1740, 1995; and WD40 is described in Hu et al., J Biol Chem., 273:33489-33494, 1998.

Additional multimerization domains and systems are described in, for example, Hodneland, et al. Proc Natl Acd Sci USA. 2002; 99: 5048-5052; Arakawa et al., J Biol. Chem., 269:27833-27839, 1994; Radziejewski et al., Biochem, 32: 1350, 1993; WO2012001647A2; U.S. Pat. No. 5,821,333; GenBank Accession no. AAF73912.1 (Nishi et al., Mol Cell Biol, 25: 2607-2621, 2005), the SH3 domain of I1l from GenBank Accession no. AAD22543.1 (Kristensen el al., EMBO J., 25: 785-797, 2006), the PTB domain of human DOK-7 from GenBank Accession no. NP_005535.1 (Wagner et al., Cold Spring Harb Perspect Biol. 5: a008987, 2013), the PDZ-like domain of SATB1 from UniProt Accession No. Q01826 (Galande et al., Mol Cell Biol. August; 21: 5591-5604, 2001), the WD40 repeats of APAF from UniProt Accession No. 014727 (Jorgensen et al., 2009. PLOS One. 4(12):e8463), the PAS motif of the dioxin receptor from UniProt Accession No. 16L9E7 (Pongratz et al., Mol Cell Biol, 18:4079-4088, 1998) and the EF hand motif of parvalbumin from UniProt Accession No. P20472 (Jamalian et al., Int J Proteomics, 2014: 153712, 2014). C4b, dextrameric, and ferritin-based multimerization can be used.

In particular embodiments, complementary binding domains can be induced using a third molecule or chemical inducer. This method of dimerization requires that one antibody or fragment thereof include a chemical inducer of dimerization binding domain 1 (CBD1) and the second antibody or fragment thereof include the second chemical inducer of dimerization binding domain (CBD2), wherein CBD1 and CBD2 are capable of simultaneously binding to a chemical inducer of dimerization (CID). CBD1 may include a rapamycin binding domain of FK-binding protein 12 (FKBP12) and CBD2 may include a FKBP12-Rapamycin Binding (FRB) domain of mTOR.

(III) Expression of Recombinant Antibodies. Antibodies disclosed herein can be produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies.

In particular embodiments, mammalian cells are used as a host for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed in the art, and include CHO cell lines (e.g., DG44), various COS cell lines, HeLa cells, HEK293 cells, L cells, and non-antibody-producing myelomas including Sp2/0 and NSO. Preferably, the cells are nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. In particular embodiments, expression control sequences are promoters derived from endogenous genes, cytomegalovirus, SV40, adenovirus, and bovine papillomavirus (see Co et al., J. Immunol. 1992, 148:1149).

Once expressed, antibodies can be purified according to standard procedures of the art, including high-performance liquid chromatography (HPLC) purification, column chromatography, gel electrophoresis and the like (see generally, Scopes, Protein Purification (Springer-Verlag, NY, 1982)).

In particular embodiments, antibodies are formed using the Daedalus expression system as described in Pechman et al. (Am J Physiol 294: R1234-R1239, 2008). The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/l of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 39(21) 2011. In some instances, purification by chromatography may not be needed due to the purity of manufacture according to the methods described herein.

(iv) Antibody Conjugates. HMPV antibody conjugates include an HMPV antibody disclosed herein linked to another molecule, other than an additional binding domain. Examples of antibody conjugates include antibody immunotoxins, antibody-drug conjugates (ADCs), antibody-detectable label conjugates, antibody radioisotope conjugates, and antibody-particle conjugates.

HMPV antibody can be formed as an antibody immunotoxin. Antibody immunotoxins include an HMPV antibody disclosed herein conjugated to one or more cytotoxins (e.g., protein toxins, enzymatically active toxins of bacterial, fungal, plant, or animal origin, or fragments thereof). A toxin can be any agent that is detrimental to cells. Frequently used plant toxins are divided into two classes: (1) holotoxins (or class II ribosome inactivating proteins), such as ricin, abrin, mistletoe lectin, and modeccin, and (2) hemitoxins (class I ribosome inactivating proteins), such as pokeweed antiviral protein (PAP), saporin, Bryodin 1, bouganin, and gelonin. Commonly used bacterial toxins include diphtheria toxin (DT) and Pseudomonas exotoxin (PE). Kreitman, Current Pharmaceutical Biotechnology 2:313-325 (2001). The toxin may be obtained from essentially any source and can be a synthetic or a natural product.

In particular embodiments, antibody-drug conjugates refer to targeted molecules which combine properties of both antibodies and cytotoxic drugs (e.g., chemotherapeutic drugs) by targeting potent cytotoxic drugs to antigen-expressing cells (Teicher, B. A. (2009) Current Cancer Drug Targets 9:982-1004), thereby enhancing the therapeutic index by maximizing efficacy and minimizing off-target toxicity (Carter, P. J. and Senter P. D. (2008) The Cancer Jour. 14(3):154-169; Chari, R. V. (2008) Acc. Chem. Res. 41:98-107). See also Kamath & Iyer (Pharm Res. 32(11): 3470-3479, 2015), which describes considerations for the development of antibody-drug conjugates. The drug moiety (D) of an antibody-drug conjugate may include any compound, moiety or group that has a cytotoxic or cytostatic effect. To prepare antibody-drug conjugates, linker-cytotoxin conjugates can be made by conventional methods analogous to those described by Doronina et al. (Bioconjugate Chem. 17: 114-124, 2006).

Antibody-detectable label conjugates include an HMPV antibody linked to a detectable label. Detectable labels can include any suitable label or detectable group detectable by, for example, optical, spectroscopic, photochemical, biochemical, immunochemical, electrical, optical or chemical means. In particular embodiments, detectable labels can include fluorescent labels (e.g., GFP, EGFP, YFP), chemiluminescent labels (e.g., luminol, imidazole), spectral colorimetric labels (e.g., colloidal gold), enzymatic labels (e.g., horseradish peroxidase, acetylcholinesterase), and affinity tags (e.g., His tag, Flag tag, Myc tag, Strep tag).

Antibody-radioisotope conjugates include an HMPV antibody linked to a radioisotope for use in nuclear medicine. Nuclear medicine refers to the diagnosis and/or treatment of conditions by administering radioactive isotopes (radioisotopes or radionuclides) to a subject. Therapeutic nuclear medicine is often referred to as radiation therapy or radioimmunotherapy (RIT). In particular embodiments, radioactive isotope include iodine-131 yttrium-90, arsenic-72, arsenic-74, iodine-131, indium-111, and lutetium-177, as well as alpha-emitting radionuclides such as astatine-211, actinium-225, bismuth-212 or bismuth-213.

Antibody-particle conjugates include an antibody linked to a particle. In particular embodiments, particles include microparticles, nanoparticles, nanoshells, nanobeads, microbeads, or nanodots. Particles can include, for example, latex beads, polystyrene beads, fluorescent beads, and/or colored beads, and can be made from organic matter and/or inorganic matter.

In particular embodiments, an antibody as disclosed herein can be linked to a conjugate by any method known in the art. In particular embodiments, the constant region can be modified to allow for site specific conjugation. Such techniques include the use of naturally occurring or engineered cysteine residues, disulfide bridges, poly-histidine sequences, glycoengineering tags, and transglutaminase recognition sequences. Antibody fragments can also be modified for site-specific conjugation, see for example, Kim et al., Mol Cancer Ther 2008; 7(8).

### (v) Recombinant Receptors

HMPV antibodies disclosed herein can be utilized within recombinant receptors such as chimeric antigen receptors (CAR) and/or engineered T cell receptors (eTCR).

CAR include several distinct subcomponents that allow genetically modified cells (e.g., regulatory T cells) to recognize and kill HMPV cells. The subcomponents include at least an extracellular component and an intracellular component. The extracellular component includes a binding domain that specifically binds an HMPV epitope that is preferentially present on the surface of cells or in the area thereof. When the binding domain binds such epitopes, the intracellular component activates the cell to destroy the bound cell. CAR additionally include a transmembrane domain that directly or indirectly links the extracellular component to the intracellular component, and other subcomponents that can increase the CAR's function. For example, the inclusion of a spacer region and/or one or more linker sequences can allow the CAR to have additional conformational flexibility, often increasing the binding domain's ability to bind the targeted epitope.

eTCR disclosed herein include an HMPV antibody disclosed herein linked to the C_{α} and/or C_{β} chains of a TCR. A TCR is a heterodimeric fusion protein that typically includes an α and β chain. Each chain includes a variable region (V_{α} and V_{β}) and a constant region (C_{α} and C_{β}). In particular embodiments, an eTCR does not include the native TCR variable region but does include the native TCR constant region. In particular embodiments, the eTCR includes an HMPV antibody as the variable region of the α and β chain. In particular embodiments, eTCR include a C_{α} and/or C_{β} chain sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a known or identified TCR C_{α} or C_{β}.

In particular embodiments, the extracellular component of a recombinant receptor includes a binding domain that binds HMPV. Particular embodiments of binding domains include an HMPV antibody and/or the CDRs thereof as disclosed herein.

Recombinant receptors can additionally include spacer regions, transmembrane domains, intracellular effector domains, transduction markers, and tags.

Spacer regions are used to create appropriate distances and/or flexibility between subcomponents of a protein. Spacer regions typically include 10 to 250 amino acids, 10 to 200 amino acids, 10 to 150 amino acids, 10 to 100 amino acids, 10 to 50 amino acids, or 10 to 25 amino acids. Exemplary spacer regions include all or a portion of an immunoglobulin hinge region.

Transmembrane domains typically have a three-dimensional structure that is thermodynamically stable in a cell membrane, and generally ranges in length from 15 to 30 amino acids. The structure of a transmembrane domain can include an α helix, a β barrel, a β sheet, a β helix, or any combination thereof. Transmembrane domains can include at least the transmembrane region(s) of the α, β or ζ chain of a T-cell receptor, CD28, CD27, CD3, CD45, CD4, CD5, CD8, CD9, CD16, CD22, CD45, CD37, CD64, CD80, CD86, CD134, CD137 and CD154.

A transmembrane domain can include one or more additional amino acids adjacent to the transmembrane region, e.g., one or more amino acid within the extracellular region of the expressed protein (e.g., up to 15 amino acids of the extracellular region) and/or one or more additional amino acids within the intracellular region of the expressed protein (e.g., up to 15 amino acids of the intracellular components).

Intracellular effector domains activate the expressing cell when the binding domain binds the antigen. The term “effector domain” is thus meant to include any portion of the intracellular domain sufficient to transduce an activation signal.

An effector domain can include one, two, three or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CD3γ, CD3δ, CD3ε, CD3ζ, CD27, CD28, DAP10, ICOS, LAG3, NKG2D, NOTCH1, OX40, ROR2, SLAMF1, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), LIGHT, NKG2C, B7-H3, a ligand that specifically binds with CD83, CDS, ICAM-1, GITR, BAFFR, SLAMF7, NKp80 (KLRF1), CD127, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (FcεRib), DAP10, and DAP12. In particular embodiments, variants of CD3ζ (retain at least one, two, three, or all ITAM regions.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD137), OX40, PD-1, ICOS, lymphocyte function-associated antigen-1 (LFA-1), NKG2C, and a ligand that specifically binds with CD83.

Transduction markers may be selected from, for example, at least one of a truncated CD19 (tCD19; see Budde et al., Blood 122: 1660, 2013); a truncated human EGFR (tEGFR; see Wang et al., Blood 118: 1255, 2011); an extracellular domain of human CD34; and/or RQR8 which combines target epitopes from CD34 (see Fehse et al, Mol. Therapy 1(5 Pt 1); 448-456, 2000) and CD20 antigens (see Philip et al, Blood 124: 1277-1278). Methods to genetically modify cells to express CAR are well-known in the art.

Recombinant receptors can additionally include tags, such as the affinity tags elsewhere herein.

### (vi) Compositions and Formulations

Any of the antibodies described herein in any exemplary format can be formulated alone or in combination into compositions for administration to subjects. Additionally, nucleic acids encoding the antibodies can also be formulated into compositions for administration (e.g., nucleic acids encapsulated within nanoparticles (e.g., liposomes or polymer-based nanoparticles) and/or as part of a vector delivery system (e.g., a viral vector or plasmid). Antibodies and/or nucleic acids encoding antibodies are collectively referred to herein as “active ingredients”. Certain examples may include formulations. Formulations include cells genetically modified to express an antibody disclosed herein within a pharmaceutically acceptable carrier.

Salts and/or pro-drugs of the active ingredients can also be used.

A pharmaceutically acceptable salt includes any salt that retains the activity of the active ingredient and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage or by hydrolysis of a biologically labile group.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA (ethylene-diamine-tetra-acetic acid).

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the antibodies or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The formulations and/or compositions disclosed herein can further be formulated for intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous administration.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the composition can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid compositions such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of the composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Additionally, compositions can be formulated as sustained-release systems utilizing semipermeable matrices of solid polymers including at least one type of antibody. Various sustained-release materials have been established and are well known by those of ordinary skill in the art. Sustained-release systems may, depending on their chemical nature, release one or more antibodies following administration for a few weeks up to over 100 days. Depot preparations can be administered by injection; parenteral injection; instillation; or implantation into soft tissues, a body cavity, or occasionally into a blood vessel with injection through fine needles.

Depot compositions can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of nanoparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot compositions can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

In certain examples, cells are genetically modified to express an antibody disclosed herein, for example, as part of a recombinant receptor including a disclosed antibody or fragment thereof (as part of, for example, a CAR or eTCR). In these embodiments, genetically modified cells can be prepared as formulations for delivery in buffers such as Hanks' solution, Ringer's solution, or physiological saline. Cells can be genetically modified using methods known in the art. Exemplary targeted genetic engineering approaches include the use of CRISPR/Cas nuclease systems, zinc finger nucleases (ZFNs), and/or transcription activator like effector nucleases (TALENs). In particular embodiments, the cells are B cells genetically modified to express an antibody. Methods to genetically modify a B cell to express an antibody are described in PCT/US2018/056789.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

In particular embodiments, cells are in a formulation volume of a liter or less, 500 ml or less, 250 ml or less, or 100 ml or less. Hence, the density of administered cells is typically greater than 10⁴ cells/ml, 10⁵ cells/ml, 10⁶ cells/ml, 10⁷ cells/ml, or 10⁸ cells/ml.

Any composition or formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers are disclosed in Remington's Pharmaceutical Sciences, 18th Ed. Mack Printing Company, 1990. Moreover, compositions can be prepared to meet sterility, pyrogenicity, general safety, and purity standards as required by U.S. FDA Office of Biological Standards and/or other relevant foreign regulatory agencies.

### (vii) Kits

Also provided herein are kits including at least one antibody or sequences encoding at least one antibody disclosed herein. Kits may be formed with components to practice, for example, the methods described herein. In particular embodiments, the kit includes an HMPV antibody, a multi-domain binding molecule, or an antibody conjugate, or sequences encoding an antibody, a multi-domain binding molecule, an or an antibody conjugate as described herein. In particular embodiments, the kit includes cells expressing a recombinant receptor or formulation to modify cells to express a recombinant receptor. The kit may include material(s), which may be desirable from a user standpoint, such as a buffer(s), a diluent(s), a standard(s), and/or other material useful in sample processing, washing, or conducting any other step of the method described herein.

The kit according to the present disclosure may also include instructions for carrying out the method. Instructions included in the kit of the present disclosure may be affixed to packaging material or may be included as a package insert. While instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site which provides instructions.

### (viii) Methods of Use

Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions and/or formulations disclosed herein. Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of an infection's development, progression, and/or resolution.

A “prophylactic treatment” includes a treatment administered to a subject who does not display signs or symptoms of an infection or displays only early signs or symptoms of an infection such that treatment is administered for the purpose of diminishing or decreasing the risk of developing the infection further. Thus, a prophylactic treatment functions as a preventative treatment against an infection. In particular embodiments, prophylactic treatments reduce, delay, or prevent the worsening of an infection.

A “therapeutic treatment” includes a treatment administered to a subject who displays symptoms or signs of an infection and is administered to the subject for the purpose of diminishing or eliminating those signs or symptoms of the infection. The therapeutic treatment can reduce, control, or eliminate the presence or activity of the infection and/or reduce control or eliminate side effects of the infection.

Function as an effective amount, prophylactic treatment or therapeutic treatment are not mutually exclusive, and in particular embodiments, administered dosages may accomplish more than one treatment type.

In particular embodiments, therapeutically effective amounts provide anti-infection effects. Anti-infection effects include a reducing or preventing a virus from infecting a cell, decreasing the number of infected cells, decreasing the volume of infected tissue, increasing lifespan, increasing life expectancy, reducing or eliminating infection-associated symptoms. In particular embodiments, therapeutically effective amounts induce an immune response. The immune response can be against HMPV infection.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of infection, stage of infection, previous or concurrent therapeutic interventions, idiopathy of the subject and route of administration.

Useful doses can range from 0.1 to 5 μg/kg or from 0.5 to 1 μg/kg. In other examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Exemplary doses of cell-based formulations can include 10⁴ to 10⁹ cells/kg body weight, or 10³ to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹ cells.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular embodiments, the treatment protocol may be dictated by a clinical trial protocol or an FDA-approved treatment protocol.

The compositions described herein can be administered by, for example, injection, inhalation, infusion, perfusion, lavage, or ingestion. Routes of administration can include intravenous, intradermal, intraarterial, intranodal, intravesicular, intrathecal, intraperitoneal, intraparenteral, intranasal, intralesional, intramuscular, oral, subcutaneous, and/or sublingual administration.

The Exemplary Embodiments below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (ix) Exemplary Embodiments

1. An antibody or binding fragment thereof that binds human metapneumovirus (HMPV) including
a variable heavy chain including a complementarity determining region (CDR) heavy (H)1, CDRH2, and CDRH3 and
a variable light chain including a CDR light (L)1, CDRL2, and CDRL3,
wherein the CDRH1, CDRH2, CDRH3, CDRL1, CDRL2, and CDRL3 are the CDRs of antibody 4F11, 4E11, 3B5, or 7F8.
2. The antibody or fragment of embodiment 1, wherein the CDRH1 includes the sequence as set forth in SEQ ID NO: 20, the CDRH2 includes the sequence as set forth in SEQ ID NO: 21, and the CDRH3 includes the sequence as set forth in SEQ ID NO: 22, the CDRL1 includes the sequence as set forth in SEQ ID NO: 23 the CDRL2 includes LGS, and the CDRL3 includes the sequence as set forth in SEQ ID NO: 24.
3. The antibody of fragment of embodiment 1, wherein the CDRH1 includes the sequence as set forth in SEQ ID NO: 1, the CDRH2 includes the sequence as set forth in SEQ ID NO: 2, and the CDRH3 includes the sequence as set forth in SEQ ID NO: 3, the CDRL1 includes the sequence as set forth in SEQ ID NO: 4, the CDRL2 includes QDN, and the CDRL3 includes the sequence as set forth in SEQ ID NO: 5.
4. The antibody or fragment of embodiment 1, wherein the CDRH1 includes the sequence as set forth in SEQ ID NO: 10, the CDRH2 includes the sequence as set forth in SEQ ID NO: 11, and the CDRH3 includes the sequence as set forth in SEQ ID NO: 12, the CDRL1 includes the sequence as set forth in SEQ ID NO: 13, the CDRL2 includes the sequence as set forth in SEQ ID NO: 14, and the CDRL3 includes the sequence as set forth in SEQ ID NO: 15.
5. The antibody or fragment of embodiment 1, wherein the CDRH1 includes the sequence as set forth in SEQ ID NO: 94, the CDRH2 includes the sequence as set forth in SEQ ID NO: 95, and the CDRH3 includes the sequence as set forth in SEQ ID NO: 96, the CDRL1 includes the sequence as set forth in SEQ ID NO: 97, the CDRL2 includes the sequence as set forth in SEQ ID NO: 98, and the CDRL3 includes the sequence as set forth in SEQ ID NO: 99.
6. The antibody or fragment thereof of any of embodiments 1-5, wherein
   the variable heavy chain has at least 90% sequence identity to the sequence as set forth in SEQ ID NO: 25 and the variable light chain has at least 90% sequence identity to the sequence as set forth in SEQ ID NO: 26;
   the variable heavy chain has at least 90% sequence identity to the sequence as set forth in SEQ ID NO: 6 and the variable light chain has at least 90% sequence identity to the sequence as set forth in SEQ ID NO: 7;
   the variable heavy chain has at least 90% sequence identity to the sequence as set forth in SEQ ID NO: 16 and the variable light chain has at least 90% sequence identity to the sequence as set forth in SEQ ID NO: 17; or
   the variable heavy chain has at least 90% sequence identity to the sequence as set forth in SEQ ID NO: 74 and the variable light chain has at least 90% sequence identity to the sequence as set forth in SEQ ID NO: 75.
7. The antibody or fragment thereof of any of embodiments 1-6, wherein
   the variable heavy chain has at least 95% sequence identity to the sequence as set forth in SEQ ID NO: 25 and the variable light chain has at least 95% sequence identity to the sequence as set forth in SEQ ID NO: 26;
   the variable heavy chain has at least 95% sequence identity to the sequence as set forth in SEQ ID NO: 6 and the variable light chain has at least 95% sequence identity to the sequence as set forth in SEQ ID NO: 7;
   the variable heavy chain has at least 95% sequence identity to the sequence as set forth in SEQ ID NO: 16 and the variable light chain has at least 95% sequence identity to the sequence as set forth in SEQ ID NO: 17; or
   the variable heavy chain has at least 95% sequence identity to the sequence as set forth in SEQ ID NO: 74 and the variable light chain has at least 95% sequence identity to the sequence as set forth in SEQ ID NO: 75.
8. The antibody or fragment thereof of any of embodiments 1-7, wherein
   the variable heavy chain has at least 99% sequence identity to the sequence as set forth in SEQ ID NO: 25 and the variable light chain has at least 99% sequence identity to the sequence as set forth in SEQ ID NO: 26;
   the variable heavy chain has at least 99% sequence identity to the sequence as set forth in SEQ ID NO: 6 and the variable light chain has at least 99% sequence identity to the sequence as set forth in SEQ ID NO: 7;
   the variable heavy chain has at least 99% sequence identity to the sequence as set forth in SEQ ID NO: 16 and the variable light chain has at least 99% sequence identity to the sequence as set forth in SEQ ID NO: 17; or
   the variable heavy chain has at least 99% sequence identity to the sequence as set forth in SEQ ID NO: 74 and the variable light chain has at least 99% sequence identity to the sequence as set forth in SEQ ID NO: 75.
9. The antibody or fragment thereof of any of embodiments 1-8, wherein
   the variable heavy chain has the sequence as set forth in SEQ ID NO: 27 and the variable light chain has the sequence as set forth in SEQ ID NO: 28;
   the variable heavy chain has the sequence as set forth in SEQ ID NO: 6 and the variable light chain has the sequence as set forth in SEQ ID NO: 7;
   the variable heavy chain has the sequence as set forth in SEQ ID NO: 16 and the variable light chain has the sequence as set forth in SEQ ID NO: 17; or
   the variable heavy chain has the sequence as set forth in SEQ ID NO: 76 and the variable light chain has the sequence as set forth in SEQ ID NO: 77.
10. The antibody or fragment thereof of any of embodiments 1-9, wherein
   the variable heavy chain is encoded by a sequence having at least 90% sequence identity to the sequence as set forth in SEQ ID NO: 27 and the variable light chain is encoded by a sequence having at least 90% sequence identity to the sequence as set forth in SEQ ID NO: 28;
   the variable heavy chain is encoded by a sequence having at least 90% sequence identity to the sequence as set forth in SEQ ID NO: 8 and the variable light chain is encoded by a sequence having at least 90% sequence identity to the sequence as set forth in SEQ ID NO: 9;
   the variable heavy chain is encoded by a sequence having at least 90% sequence identity to the sequence as set forth in SEQ ID NO: 18 and the variable light chain is encoded by a sequence having at least 90% sequence identity to the sequence as set forth in SEQ ID NO: 19; or
   the variable heavy chain is encoded by a sequence having at least 90% sequence identity to the sequence as set forth in SEQ ID NO: 76 and the variable light chain is encoded by a sequence having at least 90% sequence identity to the sequence as set forth in SEQ ID NO: 77.
11. The antibody or fragment thereof of any of embodiments 1-10, wherein
   the variable heavy chain is encoded by a sequence having at least 95% sequence identity to the sequence as set forth in SEQ ID NO: 27 and the variable light chain is encoded by a sequence having at least 95% sequence identity to the sequence as set forth in SEQ ID NO: 28;
   the variable heavy chain is encoded by a sequence having at least 95% sequence identity to the sequence as set forth in SEQ ID NO: 8 and the variable light chain is encoded by a sequence having at least 95% sequence identity to the sequence as set forth in SEQ ID NO: 9;
   the variable heavy chain is encoded by a sequence having at least 95% sequence identity to the sequence as set forth in SEQ ID NO: 18 and the variable light chain is encoded by a sequence having at least 95% sequence identity to the sequence as set forth in SEQ ID NO: 19; or
   the variable heavy chain is encoded by a sequence having at least 95% sequence identity to the sequence as set forth in SEQ ID NO: 76 and the variable light chain is encoded by a sequence having at least 95% sequence identity to the sequence as set forth in SEQ ID NO: 77.
12. The antibody or fragment thereof of any of embodiments 1-11, wherein
   the variable heavy chain is encoded by a sequence having at least 99% sequence identity to the sequence as set forth in SEQ ID NO: 27 and the variable light chain is encoded by a sequence having at least 99% sequence identity to the sequence as set forth in SEQ ID NO: 28;
   the variable heavy chain is encoded by a sequence having at least 99% sequence identity to the sequence as set forth in SEQ ID NO: 8 and the variable light chain is encoded by a sequence having at least 99% sequence identity to the sequence as set forth in SEQ ID NO: 7;
   the variable heavy chain is encoded by a sequence having at least 99% sequence identity to the sequence as set forth in SEQ ID NO: 18 and the variable light chain is encoded by a sequence having at least 99% sequence identity to the sequence as set forth in SEQ ID NO: 19; or
   the variable heavy chain is encoded by a sequence having at least 99% sequence identity to the sequence as set forth in SEQ ID NO: 76 and the variable light chain is encoded by a sequence having at least 99% sequence identity to the sequence as set forth in SEQ ID NO: 77.
13. The antibody or fragment thereof of any of embodiments 1-12, wherein
   the variable heavy chain is encoded by a sequence having the sequence as set forth in SEQ ID NO: 27 and the variable light chain is encoded by a sequence having the sequence as set forth in SEQ ID NO: 28;
   the variable heavy chain is encoded by a sequence having the sequence as set forth in SEQ ID NO: 8 and the variable light chain is encoded by a sequence having the sequence as set forth in SEQ ID NO: 9;
   the variable heavy chain is encoded by a sequence having the sequence as set forth in SEQ ID NO: 18 and the variable light chain is encoded by a sequence having the sequence as set forth in SEQ ID NO: 19; or
   the variable heavy chain is encoded by a sequence having the sequence as set forth in SEQ ID NO: 76 and the variable light chain is encoded by a sequence having the sequence as set forth in SEQ ID NO: 77.
14. The antibody or fragment thereof of any of embodiments 1-13, wherein the antibody or fragment thereof further includes a human constant region.
15. The antibody or fragment thereof of embodiment 14, wherein the human constant region includes a human light chain constant region and/or a human heavy chain constant region.
16. The antibody or fragment thereof of embodiment 15, wherein the human light chain constant region includes a human IgK light chain constant region or a human Igλ light chain constant region.
17. The antibody or fragment thereof of embodiment 16, wherein the human IgK light chain constant region includes the sequence as set forth in SEQ ID NO: 29.
18. The antibody or fragment thereof of embodiment 16, wherein the human Igλ light chain constant region includes the sequence as set forth in SEQ ID NO: 30.
19. The antibody or fragment thereof of any of embodiments 1-18, including a heavy chain variable region including the sequence as set forth in SEQ ID NO: 25, a light chain variable region including the sequence as set forth in SEQ ID NO: 26, an IgG human heavy chain constant region, and a human IgK light chain constant region.
20. The antibody or fragment thereof of any of embodiments 1-18, including a heavy chain variable region encoded by the sequence as set forth in SEQ ID NO: 27, a light chain variable region encoded by the sequence as set forth in SEQ ID NO: 28, an IgG human heavy chain constant region, and a human IgK light chain constant region.
21. The antibody or fragment thereof of any of embodiments 1-18, including a heavy chain variable region including the sequence as set forth in SEQ ID NO: 6, a light chain variable region including the sequence as set forth in SEQ ID NO: 7, an IgG human heavy chain constant region, and a human Igλ light chain constant region.
22. The antibody or fragment thereof of any of embodiments 1-18, including a heavy chain variable region encoded by the sequence as set forth in SEQ ID NO: 8, a light chain variable region encoded by the sequence as set forth in SEQ ID NO: 9, an IgG human heavy chain constant region, and a human Igλ light chain constant region.
23. The antibody or fragment thereof of any of embodiments 1-18, including a heavy chain variable region including the sequence as set forth in SEQ ID NO: 16, a light chain variable region including the sequence as set forth in SEQ ID NO: 17, an IgG human heavy chain constant region, and a human Igλ light chain constant region.
24. The antibody or fragment thereof of any of embodiments 1-18, including a heavy chain variable region encoded by the sequence as set forth in SEQ ID NO: 18, a light chain variable region encoded by the sequence as set forth in SEQ ID NO: 19, an IgG human heavy chain constant region, and a human Igλ light chain constant region.
25. The antibody or fragment thereof of any of embodiments 1-18, including a heavy chain variable region including the sequence as set forth in SEQ ID NO: 74, a light chain variable region including the sequence as set forth in SEQ ID NO: 75, an IgG human heavy chain constant region, and a human Igλ light chain constant region.
26. The antibody or fragment thereof of any of embodiments 1-18, including a heavy chain variable region encoded by the sequence as set forth in SEQ ID NO: 76, a light chain variable region encoded by the sequence as set forth in SEQ ID NO: 77, an IgG human heavy chain constant region, and a human IgA light chain constant region.
27. A multi-domain binding molecule including at least two binding domains, wherein at least one binding domain of the at least two binding domains includes the antibody or fragment thereof of any of embodiments 1-26.
28. The multi-domain binding molecule of embodiment 27, wherein the multi-domain binding molecule includes an immune cell engaging molecule.
29. The multi-domain binding molecule of embodiment 28, wherein the immune cell engaging molecule activates a B cell, T cell, natural killer (NK) cell, or macrophage.
30. The multi-domain binding molecule of embodiment 29, wherein the T cell is a CD3 T cell, a CD4 T cell, a CD8 T cell, a central memory T cell, an effector memory T cell, and/or a naive T cell.
31. The multi-domain binding molecule of any of embodiments 28-30, wherein a binding domain of the immune cell engaging molecule binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Rα, or MARCO.
32. The multi-domain binding molecule of any of embodiments 27-31, wherein the at least two binding domains include at least two copies of the antibody or fragment thereof of any of embodiments 1-26.
33. The multi-domain binding molecule of embodiment 32, wherein the at least two copies are joined by a protein linker.
34. The multi-domain binding molecule of embodiment 33, wherein the protein linker is a Gly-Ser linker.
35. The multi-domain binding molecule of embodiment 34, wherein the Gly-Ser linker is (GlyxSery)ₙ wherein x and y are independently an integer from 0 to 10 provided that x and y are not both 0 and wherein n is an integer of 1, 2, 3, 4, 5, 6, 7, 8, 9 or 10.
36. The multi-domain binding molecule of any of embodiments 27-35, including 2, 3, 4, 5, 6, 7, 8, 9, or 10 copies of the antibody or fragment thereof of any of embodiments 1-26.
37. The multi-domain binding molecule of any of embodiments 27-36, wherein the multi-domain binding molecule is a dimer, trimer, tetramer, pentamer, hexamer, or heptamer.
38. The multi-domain binding molecule of any of embodiments 33-37, wherein the at least two copies are linked to an Fc region of an antibody.
39. The multi-domain binding molecule of embodiment 38, wherein the Fc region is an IgA Fc region or an IgM Fc region.
40. The multi-domain binding molecule of embodiment 39, wherein the Fc region is an IgA Fc region having the sequence as set forth in SEQ ID NOs: 50 and 51.
41. The multi-domain binding molecule of any of embodiments 39-40, wherein the Fc region includes a multimerizing fragment of the IgA Fc region or a multimerizing fragment of the IgM Fc region.
42. The multi-domain binding molecule of embodiment 42, wherein the multimerizing fragment of the IgM Fc region has the sequence as set forth in SEQ ID NO: 55.
43. A composition including an antibody or fragment thereof of any of embodiments 1-26 and a pharmaceutically-acceptable carrier.
44. The composition of embodiment 54, wherein the composition includes a therapeutically-effective amount of the antibody or fragment thereof for administration to a subject.
45. A formulation including a cell genetically modified to express an antibody or fragment thereof of any of embodiments 1-26 and a pharmaceutically-acceptable carrier.
46. A method of providing an anti-human metapneumovirus (HMPV) effect in a subject in need thereof including administering a therapeutically effective amount of the composition of embodiments 54 or 55 or the formulation of embodiment 56 to the subject thereby providing the anti-HMPV effect to the subject in need thereof.
47. The method of embodiment 57, wherein the anti-viral effect further includes an anti-viral effect against a secondary virus.
48. The method of embodiment 58, wherein the secondary virus is selected from an adenovirus, a boca virus, a coronavirus, an enterovirus, an influenza virus, a metapneumovirus, a parainfluenza virus, a respiratory syncytial virus (RSV), and/or a rhinovirus.

### (x) Closing Paragraphs

The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (nonpolar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein. [0214]“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding fragment (of, for example, a binding fragment) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding fragments may be classified as “high affinity” or “low affinity”. In particular embodiments, “high affinity” binding fragments refer to those binding fragments with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding fragments refer to those binding fragments with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding fragment may have “enhanced affinity,” which refers to a selected or engineered binding fragments with stronger binding to a cognate binding molecule than a wild type (or parent) binding fragment. For example, enhanced affinity may be due to a Ka (equilibrium association constant) for the cognate binding molecule that is higher than the reference binding fragment or due to a Kd (dissociation constant) for the cognate binding molecule that is less than that of the reference binding fragment, or due to an off-rate (Koff) for the cognate binding molecule that is less than that of the reference binding fragment. A variety of assays are known for detecting binding fragments that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in binding between a disclosed antibody and its viral epitope.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; 19% of the stated value; ±18% of the stated value; 17% of the stated value; 16% of the stated value; ±15% of the stated value; 14% of the stated value; ±13% of the stated value; 12% of the stated value; 11% of the stated value; 10% of the stated value; 9% of the stated value; 8% of the stated value; 7% of the stated value; ±6% of the stated value; 5% of the stated value; 4% of the stated value; ±3% of the stated value; 2% of the stated value; or +1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. An antibody or binding fragment thereof that binds human metapneumovirus (HMPV) comprising
a variable heavy chain comprising a complementarity determining region (CDR) heavy (H)1, CDRH2, and CDRH3 and
a variable light chain comprising a CDR light (L)1, CDRL2, and CDRL3,
wherein the CDRH1, CDRH2, CDRH3, CDRL1, CDRL2, and CDRL3 are the CDRs of antibody 4F11, 4E11, 3B5, or 7F8.

2. The antibody or fragment of claim 1, wherein the CDRH1 comprises the sequence as set forth in SEQ ID NO: 20, the CDRH2 comprises the sequence as set forth in SEQ ID NO: 21, and the CDRH3 comprises the sequence as set forth in SEQ ID NO: 22, the CDRL1 comprises the sequence as set forth in SEQ ID NO: 23 the CDRL2 comprises LGS, and the CDRL3 comprises the sequence as set forth in SEQ ID NO: 24.

3. The antibody or fragment of claim 1, wherein the CDRH1 comprises the sequence as set forth in SEQ ID NO: 1, the CDRH2 comprises the sequence as set forth in SEQ ID NO: 2, and the CDRH3 comprises the sequence as set forth in SEQ ID NO: 3, the CDRL1 comprises the sequence as set forth in SEQ ID NO: 4, the CDRL2 comprises QDN, and the CDRL3 comprises the sequence as set forth in SEQ ID NO: 5.

4. The antibody or fragment of claim 1, wherein the CDRH1 comprises the sequence as set forth in SEQ ID NO: 10, the CDRH2 comprises the sequence as set forth in SEQ ID NO: 11, and the CDRH3 comprises the sequence as set forth in SEQ ID NO: 12, the CDRL1 comprises the sequence as set forth in SEQ ID NO: 13, the CDRL2 comprises the sequence as set forth in SEQ ID NO: 14, and the CDRL3 comprises the sequence as set forth in SEQ ID NO: 15.

5. The antibody or fragment of claim 1, wherein the CDRH1 comprises the sequence as set forth in SEQ ID NO: 94, the CDRH2 comprises the sequence as set forth in SEQ ID NO: 95, and the CDRH3 comprises the sequence as set forth in SEQ ID NO: 96, the CDRL1 comprises the sequence as set forth in SEQ ID NO: 97, the CDRL2 comprises the sequence as set forth in SEQ ID NO: 98, and the CDRL3 comprises the sequence as set forth in SEQ ID NO: 99.

6. The antibody or fragment thereof of claim 1, wherein
the variable heavy chain has at least 90% sequence identity to the sequence as set forth in SEQ ID NO: 25 and the variable light chain has at least 90% sequence identity to the sequence as set forth in SEQ ID NO: 26;
the variable heavy chain has at least 90% sequence identity to the sequence as set forth in SEQ ID NO: 6 and the variable light chain has at least 90% sequence identity to the sequence as set forth in SEQ ID NO: 7;
the variable heavy chain has at least 90% sequence identity to the sequence as set forth in SEQ ID NO: 16 and the variable light chain has at least 90% sequence identity to the sequence as set forth in SEQ ID NO: 17; or
the variable heavy chain has at least 90% sequence identity to the sequence as set forth in SEQ ID NO: 74 and the variable light chain has at least 90% sequence identity to the sequence as set forth in SEQ ID NO: 75.

7. The antibody or fragment thereof of claim 1, wherein
the variable heavy chain has at least 95% sequence identity to the sequence as set forth in SEQ ID NO: 25 and the variable light chain has at least 95% sequence identity to the sequence as set forth in SEQ ID NO: 26;
the variable heavy chain has at least 95% sequence identity to the sequence as set forth in SEQ ID NO: 6 and the variable light chain has at least 95% sequence identity to the sequence as set forth in SEQ ID NO: 7;
the variable heavy chain has at least 95% sequence identity to the sequence as set forth in SEQ ID NO: 16 and the variable light chain has at least 95% sequence identity to the sequence as set forth in SEQ ID NO: 17; or
the variable heavy chain has at least 95% sequence identity to the sequence as set forth in SEQ ID NO: 74 and the variable light chain has at least 95% sequence identity to the sequence as set forth in SEQ ID NO: 75.

8. The antibody or fragment thereof of claim 1, wherein
the variable heavy chain has at least 99% sequence identity to the sequence as set forth in SEQ ID NO: 25 and the variable light chain has at least 99% sequence identity to the sequence as set forth in SEQ ID NO: 26;
the variable heavy chain has at least 99% sequence identity to the sequence as set forth in SEQ ID NO: 6 and the variable light chain has at least 99% sequence identity to the sequence as set forth in SEQ ID NO: 7;
the variable heavy chain has at least 99% sequence identity to the sequence as set forth in SEQ ID NO: 16 and the variable light chain has at least 99% sequence identity to the sequence as set forth in SEQ ID NO: 17; or
the variable heavy chain has at least 99% sequence identity to the sequence as set forth in SEQ ID NO: 74 and the variable light chain has at least 99% sequence identity to the sequence as set forth in SEQ ID NO: 75.

9. The antibody or fragment thereof of claim 1, wherein
the variable heavy chain has the sequence as set forth in SEQ ID NO: 27 and the variable light chain has the sequence as set forth in SEQ ID NO: 28;
the variable heavy chain has the sequence as set forth in SEQ ID NO: 6 and the variable light chain has the sequence as set forth in SEQ ID NO: 7;
the variable heavy chain has the sequence as set forth in SEQ ID NO: 16 and the variable light chain has the sequence as set forth in SEQ ID NO: 17; or
the variable heavy chain has the sequence as set forth in SEQ ID NO: 76 and the variable light chain has the sequence as set forth in SEQ ID NO: 77.

10. The antibody or fragment thereof of claim 1, wherein
the variable heavy chain is encoded by a sequence having at least 90% sequence identity to the sequence as set forth in SEQ ID NO: 27 and the variable light chain is encoded by a sequence having at least 90% sequence identity to the sequence as set forth in SEQ ID NO: 28;
the variable heavy chain is encoded by a sequence having at least 90% sequence identity to the sequence as set forth in SEQ ID NO: 8 and the variable light chain is encoded by a sequence having at least 90% sequence identity to the sequence as set forth in SEQ ID NO: 9;
the variable heavy chain is encoded by a sequence having at least 90% sequence identity to the sequence as set forth in SEQ ID NO: 18 and the variable light chain is encoded by a sequence having at least 90% sequence identity to the sequence as set forth in SEQ ID NO: 19; or
the variable heavy chain is encoded by a sequence having at least 90% sequence identity to the sequence as set forth in SEQ ID NO: 76 and the variable light chain is encoded by a sequence having at least 90% sequence identity to the sequence as set forth in SEQ ID NO: 77.

11. The antibody or fragment thereof of claim 1, wherein
the variable heavy chain is encoded by a sequence having at least 95% sequence identity to the sequence as set forth in SEQ ID NO: 27 and the variable light chain is encoded by a sequence having at least 95% sequence identity to the sequence as set forth in SEQ ID NO: 28;
the variable heavy chain is encoded by a sequence having at least 95% sequence identity to the sequence as set forth in SEQ ID NO: 8 and the variable light chain is encoded by a sequence having at least 95% sequence identity to the sequence as set forth in SEQ ID NO: 9;
the variable heavy chain is encoded by a sequence having at least 95% sequence identity to the sequence as set forth in SEQ ID NO: 18 and the variable light chain is encoded by a sequence having at least 95% sequence identity to the sequence as set forth in SEQ ID NO: 19; or
the variable heavy chain is encoded by a sequence having at least 95% sequence identity to the sequence as set forth in SEQ ID NO: 76 and the variable light chain is encoded by a sequence having at least 95% sequence identity to the sequence as set forth in SEQ ID NO: 77.

12. The antibody or fragment thereof of claim 1, wherein
the variable heavy chain is encoded by a sequence having at least 99% sequence identity to the sequence as set forth in SEQ ID NO: 27 and the variable light chain is encoded by a sequence having at least 99% sequence identity to the sequence as set forth in SEQ ID NO: 28;
the variable heavy chain is encoded by a sequence having at least 99% sequence identity to the sequence as set forth in SEQ ID NO: 8 and the variable light chain is encoded by a sequence having at least 99% sequence identity to the sequence as set forth in SEQ ID NO: 7;
the variable heavy chain is encoded by a sequence having at least 99% sequence identity to the sequence as set forth in SEQ ID NO: 18 and the variable light chain is encoded by a sequence having at least 99% sequence identity to the sequence as set forth in SEQ ID NO: 19; or
the variable heavy chain is encoded by a sequence having at least 99% sequence identity to the sequence as set forth in SEQ ID NO: 76 and the variable light chain is encoded by a sequence having at least 99% sequence identity to the sequence as set forth in SEQ ID NO: 77.

13. The antibody or fragment thereof of claim 1, wherein
the variable heavy chain is encoded by a sequence having the sequence as set forth in SEQ ID NO: 27 and the variable light chain is encoded by a sequence having the sequence as set forth in SEQ ID NO: 28;
the variable heavy chain is encoded by a sequence having the sequence as set forth in SEQ ID NO: 8 and the variable light chain is encoded by a sequence having the sequence as set forth in SEQ ID NO: 9;
the variable heavy chain is encoded by a sequence having the sequence as set forth in SEQ ID NO: 18 and the variable light chain is encoded by a sequence having the sequence as set forth in SEQ ID NO: 19; or
the variable heavy chain is encoded by a sequence having the sequence as set forth in SEQ ID NO: 76 and the variable light chain is encoded by a sequence having the sequence as set forth in SEQ ID NO: 77.

14. The antibody or fragment thereof of claim 1, wherein the antibody or fragment thereof further comprises a human constant region.

15. The antibody or fragment thereof of claim 14, wherein the human constant region comprises a human light chain constant region and/or a human heavy chain constant region.

16. The antibody or fragment thereof of claim 15, wherein the human light chain constant region comprises a human IgK light chain constant region or a human Igλ light chain constant region.

17. The antibody or fragment thereof of claim 16, wherein the human IgK light chain constant region comprises the sequence as set forth in SEQ ID NO: 29.

18. The antibody or fragment thereof of claim 16, wherein the human Igλ light chain constant region comprises the sequence as set forth in SEQ ID NO: 30.

19. The antibody or fragment thereof of claim 1, comprising a heavy chain variable region comprising the sequence as set forth in SEQ ID NO: 25, a light chain variable region comprising the sequence as set forth in SEQ ID NO: 26, an IgG human heavy chain constant region, and a human IgK light chain constant region.

20. The antibody or fragment thereof of claim 1, comprising a heavy chain variable region encoded by the sequence as set forth in SEQ ID NO: 27, a light chain variable region encoded by the sequence as set forth in SEQ ID NO: 28, an IgG human heavy chain constant region, and a human IgK light chain constant region.

21. The antibody or fragment thereof of claim 1, comprising a heavy chain variable region comprising the sequence as set forth in SEQ ID NO: 6, a light chain variable region comprising the sequence as set forth in SEQ ID NO: 7, an IgG human heavy chain constant region, and a human Igλ light chain constant region.

22. The antibody or fragment thereof of claim 1, comprising a heavy chain variable region encoded by the sequence as set forth in SEQ ID NO: 8, a light chain variable region encoded by the sequence as set forth in SEQ ID NO: 9, an IgG human heavy chain constant region, and a human Igλ light chain constant region.

23. The antibody or fragment thereof of claim 1, comprising a heavy chain variable region comprising the sequence as set forth in SEQ ID NO: 16, a light chain variable region comprising the sequence as set forth in SEQ ID NO: 17, an IgG human heavy chain constant region, and a human Igλ light chain constant region.

24. The antibody or fragment thereof of claim 1, comprising a heavy chain variable region encoded by the sequence as set forth in SEQ ID NO: 18, a light chain variable region encoded by the sequence as set forth in SEQ ID NO: 19, an IgG human heavy chain constant region, and a human Igλ light chain constant region.

25. The antibody or fragment thereof of claim 1, comprising a heavy chain variable region comprising the sequence as set forth in SEQ ID NO: 74, a light chain variable region comprising the sequence as set forth in SEQ ID NO: 75, an IgG human heavy chain constant region, and a human Igλ light chain constant region.

26. The antibody or fragment thereof of claim 1, comprising a heavy chain variable region encoded by the sequence as set forth in SEQ ID NO: 76, a light chain variable region encoded by the sequence as set forth in SEQ ID NO: 77, an IgG human heavy chain constant region, and a human Igλ light chain constant region.

27. A multi-domain binding molecule comprising at least two binding domains, wherein at least one binding domain of the at least two binding domains comprises the antibody or fragment thereof of claim 1.

28. The multi-domain binding molecule of claim 27, wherein the multi-domain binding molecule comprises an immune cell engaging molecule.

29. The multi-domain binding molecule of claim 28, wherein the immune cell engaging molecule activates a B cell, T cell, natural killer (NK) cell, or macrophage.

30. The multi-domain binding molecule of claim 29, wherein the T cell is a CD3 T cell, a CD4 T cell, a CD8 T cell, a central memory T cell, an effector memory T cell, and/or a naive T cell.

31. The multi-domain binding molecule of claim 28, wherein a binding domain of the immune cell engaging molecule binds CD3, CD28, CD8, NKG2D, CD8, CD16, KIR2DL4, KIR2DS1, KIR2DS2, KIR3DS1, NKG2C, NKG2E, NKG2D, NKp30, NKp44, NKp46, NKp80, DNAM-1, CD11b, CD11c, CD64, CD68, CD119, CD163, CD206, CD209, F4/80, IFGR2, Toll-like receptors 1-9, IL-4Rα, or MARCO.

32. The multi-domain binding molecule of claim 27, wherein the at least two binding domains comprise at least two copies of the antibody or fragment thereof of claim 1.

33. The multi-domain binding molecule of claim 32, wherein the at least two copies are joined by a protein linker.

34. The multi-domain binding molecule of claim 33, wherein the protein linker is a Gly-Ser linker.

35. The multi-domain binding molecule of claim 34, wherein the Gly-Ser linker is (GlyxSery)ₙ wherein x and y are independently an integer from 0 to 10 provided that x and y are not both 0 and wherein n is an integer of 1, 2, 3, 4, 5, 6, 7, 8, 9 or 10.

36. The multi-domain binding molecule of claim 27, comprising 2, 3, 4, 5, 6, 7, 8, 9, or 10 copies of the antibody or fragment thereof of claim 1.

37. The multi-domain binding molecule of claim 27, wherein the multi-domain binding molecule is a dimer, trimer, tetramer, pentamer, hexamer, or heptamer.

38. The multi-domain binding molecule of claim 32, wherein the at least two copies are linked to an Fc region of an antibody.

39. The multi-domain binding molecule of claim 38, wherein the Fc region is an IgA Fc region or an IgM Fc region.

40. The multi-domain binding molecule of claim 39, wherein the Fc region is an IgA Fc region having the sequence as set forth in SEQ ID NOs: 50 and 51.

41. The multi-domain binding molecule of claim 39, wherein the Fc region comprises a multimerizing fragment of the IgA Fc region or a multimerizing fragment of the IgM Fc region.

42. The multi-domain binding molecule of claim 41, wherein the multimerizing fragment of the IgM Fe region has the sequence as set forth in SEQ ID NO: 55.

43. A composition comprising an antibody or fragment thereof of claim 1 and a pharmaceutically-acceptable carrier.

44. The composition of claim 43, wherein the composition comprises a therapeutically-effective amount of the antibody or fragment thereof for administration to a subject.

45. A formulation comprising a cell genetically modified to express an antibody or fragment thereof of claim 1 and a pharmaceutically-acceptable carrier.

46. A method of providing an anti-human metapneumovirus (HMPV) effect in a subject in need thereof comprising administering a therapeutically effective amount of the composition of claim 43. or the formulation of claim 45 to the subject thereby providing the anti-HMPV effect to the subject in need thereof.

47. The method of claim 46, wherein the anti-viral effect further comprises an anti-viral effect against a secondary virus.

48. The method of claim 47, wherein the secondary virus is selected from an adenovirus, a boca virus, a coronavirus, an enterovirus, an influenza virus, a metapneumovirus, a parainfluenza virus, a respiratory syncytial virus (RSV), and/or a rhinovirus.


SEQUENCE LISTING (101 sequences)

SEQ ID NO: 1 - protein, 8 aa, synthetic construct
GFSFSNYD

SEQ ID NO: 2 - protein, 8 aa, synthetic construct
VSFNGNTK

SEQ ID NO: 3 - protein, 20 aa, synthetic construct
ASGYCHGGLCHIQNYFAMDV

SEQ ID NO: 4 - protein, 6 aa, synthetic construct
KLGDNY

SEQ ID NO: 5 - protein, 11 aa, synthetic construct
QTWDSSIAPWV

SEQ ID NO: 6 - protein, 127 aa, synthetic construct
QVQLVQSGGGVVQPGRSLRLSCAASGFSFSNYDIHWVRQAPGKGLEWVALVSFNGNTKYYAESVTGRFTVSRDNSKDTLYLQMNSLRVEDTAVYYCASGYCHGGLCHIQNYFAMDVWGHGTTVTVSS

SEQ ID NO: 7 - protein, 108 aa, synthetic construct
SYELTQPPSVSVSPGQTARITCSRDKLGDNYVSWYQQRPGQSPLLVIFQDNKRPSGIPARFSGSNSGNTATLTISGTQAMDEADYYCQTWDSSIAPWVFGGGTKLTVL

SEQ ID NO: 8 - DNA, 381 bp, synthetic construct
  source 1..381: mol_type = other DNA
caggtgcagctggtgcagtccgggggaggggtggtccagcctgggaggtccctgagactctcttgtgcagcctctggattcagtttcagtaactatgacatacactgggtccgccaggctccagggaagggactggagtgggtggctcttgtttcttttaatgggaatacaaaatattatgcagagtccgtcacaggccgcttcaccgtctccagagacaattccaaggacacgttgtatctgcaaatgaacagcctgagagttgaggacacggctgtctattattgtgcgtcgggatattgtcatggtggtctgtgtcacatacagaactacttcgctatggacgtctggggccacgggaccacggtcacagtctcctca

SEQ ID NO: 9 - DNA, 325 bp, synthetic construct
  source 1..325: mol_type = other DNA
tcctatgagctgactcagccaccctcagtgtccgtgtccccaggacagacagccagaatcacctgctctagagataaattgggggataattatgtttcctggtatcaacagaggccaggccagtcccctctcttggtcatctttcaagataacaagcgaccctcagggatccctgcgcgattttctggctccaactctgggaacacggccactctgaccatcagcgggacccaggctatggatgaggctgactattactgtcagacgtgggacagcagcattgcgccctgggtgttcggcggagggaccaagctgaccgtcctgg

SEQ ID NO: 10 - protein, 8 aa, synthetic construct
GFIVSNNH

SEQ ID NO: 11 - protein, 7 aa, synthetic construct
LYSGGSA

SEQ ID NO: 12 - protein, 11 aa, synthetic construct
ARDLHAPDKPN

SEQ ID NO: 13 - protein, 7 aa, synthetic construct
RGHSTYA

SEQ ID NO: 14 - protein, 7 aa, synthetic construct
VNSDGRH

SEQ ID NO: 15 - protein, 10 aa, synthetic construct
QTWGTDIHVV

SEQ ID NO: 16 - protein, 117 aa, synthetic construct
EVQLVESGGGLVQPGGSLRLSCAASGFIVSNNHMTWVRQTPGKGLECVSVLYSGGSADYADSVKGRFIISRDNSKNTLYLQMNSLRAEDTAVYYCARDLHAPDKPNWGQGTLVTVSS

SEQ ID NO: 17 - protein, 112 aa, synthetic construct
QLVLTQSPSASASLGASVKLTCTLSRGHSTYAIAWHQKQPDKGPRYLMRVNSDGRHTKGGGIPDRFSGSSSGAERYLTISSLQSEDEADYYCQTWGTDIHVVFGEGTKLTVL

SEQ ID NO: 18 - DNA, 352 bp, synthetic construct
  source 1..352: mol_type = other DNA
gaggtgcagctggtggagtccgggggaggcttggtccagcctggggggtctctgagactctcctgtgcggcctctggattcatcgtcagtaacaaccacatgacctgggtccgccagactccagggaaggggctggaatgtgtctcggttctttatagtggtggtagcgcagactacgcggactccgtgaagggcagattcatcatttccagagacaattccaagaacaccctgtatcttcaaatgaacagcctgagagccgaggacacggctgtatattactgtgcgagagatcttcacgcgcccgacaaacccaattggggccagggaaccttggtcaccgtctcctcag

SEQ ID NO: 19 - DNA, 337 bp, synthetic construct
  source 1..337: mol_type = other DNA
cagcttgtgctgactcaatcgccgtctgcctctgcctccctgggagcctcggtcaagctcacctgcactctgagcaggggtcacagcacctacgccatcgcatggcatcagaagcagccagacaagggccctcggtacttgatgagagttaacagtgatggcaggcacaccaaggggggcgggatccctgatcgcttctcaggctccagctctggggctgagcgctacctcaccatctccagcctccagtctgaggatgaggctgactattactgtcagacctggggcactgacattcatgtggtgttcggcgaagggaccaagctgaccgtcctag

SEQ ID NO: 20 - protein, 8 aa, synthetic construct
GYKFATYW

SEQ ID NO: 21 - protein, 8 aa, synthetic construct
IYPDDSDT

SEQ ID NO: 22 - protein, 16 aa, synthetic construct
ARCYDFWSGYQFGMDV

SEQ ID NO: 23 - protein, 11 aa, synthetic construct
QSLRHDNGYNY

SEQ ID NO: 24 - protein, 10 aa, synthetic construct
MQTLQTLMFT

SEQ ID NO: 25 - protein, 123 aa, synthetic construct
EVQLVQSGAEVKKPGDSLKISCKGSGYKFATYWIGWVRQMPGKGLEWMGVIYPDDSDTRYSPSFQGQVSISVDKSITTAYLQWSSLKASDTAIYYCARCYDFWSGYQFGMDVWGQGTTVTVSS

SEQ ID NO: 26 - protein, 113 aa, synthetic construct
DIVMTQSPLSLPVTPGETASISCRSSQSLRHDNGYNYLDWYLQKPGQSPQLLIYLGSKRASGVPDRFSGSGSGTDFTLKISRVEAEDVGVYYCMQTLQTLMFTFGGGTKVEIK

SEQ ID NO: 27 - DNA, 369 bp, synthetic construct
  source 1..369: mol_type = other DNA
gaggtgcagctggtgcagtctggagcagaggtgaaaaagcccggtgactctctgaagatctcctgtaagggttctggatacaagtttgccacctactggatcggctgggtgcgccagatgcccgggaaaggcctggagtggatgggggtcatctatcctgatgactctgataccagatacagcccgtccttccaaggccaggtctccatctcagtcgacaaatccatcactaccgcctacttgcagtggagtagcctgaaggcctcggacaccgccatatattactgtgcgagatgctacgatttttggagtggttatcagttcggtatggacgtctggggccaggggaccacggtcaccgtctcctca

SEQ ID NO: 28 - DNA, 340 bp, synthetic construct
  source 1..340: mol_type = other DNA
gatattgtgatgactcagtctccactctccctgcccgtcactcctggagagacggcctccatctcctgcaggtctagtcagagcctccggcatgataatggatacaactatttggattggtacctgcagaagccagggcagtctccacagctcctgatctatttgggttctaagcgggcctccggggtcccagacaggttcagtggcagtggatcaggcacagactttacactgaaaatcagtagagtggaggctgaggatgttggggtttattactgcatgcaaactctacaaactctgatgttcactttcggcggagggaccaaggtggagatcaaac

SEQ ID NO: 29 - protein, 107 aa, Homo sapiens [sequence omitted]

SEQ ID NO: 30 - protein, 114 aa, Homo sapiens
SVSVSPGQTARITCSGDALPKKYAYWYQQKSGQAPVLVIYEDNRRPSGIPERFSGSSSRTLATLTISGAQVEDEADYYCYSTDSSGNHVVFGGGTKLTVLRQPKAAPSVTSVPT

SEQ ID NO: 31 - protein, 330 aa, Homo sapiens [sequence omitted]

SEQ ID NO: 32 - protein, 225 aa, Homo sapiens
THTCPPCPAPEFFGGPSVFFFPPKPKDTFMISRTPEVTCVVVDVSHEDPEVKFNWYVDGVEVHNAKTKPREEQYNSTYRVVSVETVFHQDWENGKEYKCKVSNKAFPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 33 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 218 aa, Homo sapiens
PAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 35 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 5 aa, synthetic construct
  REGION 1..5: note = The entire sequence of amino acids 1-5 can be repeated 1, 2, 3, 4, 5, 6, 7, 8, 9 or 10 times.
GGGGS

SEQ ID NO: 37 - protein, 9 aa, synthetic construct
  REGION 1..4: note = The entire sequence of amino acids 1-4 can be repeated 1, 2, 3, 4, 5, 6, 7, 8, 9 or 10 times.
  REGION 5..9: note = The entire sequence of amino acids 5-9 can be repeated 1, 2, 3, 4, 5, 6, 7, 8, 9 or 10 times.
GGGSGGGGS

SEQ ID NO: 38 - protein, 7 aa, synthetic construct
  REGION 1..4: note = The entire sequence of amino acids 1-4 can be repeated 1, 2, 3, 4, 5, 6, 7, 8, 9 or 10 times.
  REGION 5..7: note = The entire sequence of amino acids 5-7 can be repeated 1, 2, 3, 4, 5, 6, 7, 8, 9 or 10 times.
GGGSGGS

SEQ ID NO: 39 - protein, 9 aa, synthetic construct
  REGION 1..4: note = The entire sequence of amino acids 1-4 can be repeated 1, 2, 3, 4, 5, 6, 7, 8, 9 or 10 times.
GGGSGGGGS

SEQ ID NO: 40 - protein, 20 aa, synthetic construct
GGGGSGGGGSGGGGSGGGGS

SEQ ID NO: 41 - protein, 15 aa, synthetic construct
GGGGSGGGGSGGGGS

SEQ ID NO: 42 - protein, 10 aa, synthetic construct
GGGGSGGGGS

SEQ ID NO: 43 - protein, 5 aa, synthetic construct
GGGGS

SEQ ID NO: 44 - protein, 8 aa, synthetic construct
GGGSGGGS

SEQ ID NO: 45 - protein, 4 aa, synthetic construct
GGGS

SEQ ID NO: 46 - protein, 6 aa, synthetic construct
GGSGGS

SEQ ID NO: 47 - protein, 11 aa, synthetic construct
GGSGGGSGGSG

SEQ ID NO: 48 - protein, 10 aa, synthetic construct
GGSGGGSGSG

SEQ ID NO: 49 - protein, 8 aa, synthetic construct
GGSGGGSG

SEQ ID NO: 50 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 51 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 52-54 - skipped (3 entries)

SEQ ID NO: 55 - protein, 349 aa, Homo sapiens
VIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 56-57 - skipped (2 entries)

SEQ ID NO: 58 - protein, 453 aa, Homo sapiens [sequence omitted]

SEQ ID NO: 59 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 60-71 - skipped (12 entries)

SEQ ID NO: 72 - protein, 5 aa, synthetic construct
  REGION 1..5: note = The entire sequence of amino acids 1-5 can be repeated 1, 2, 3, 4, or 5 times
GGGGS

SEQ ID NO: 73 - skipped

SEQ ID NO: 74 - protein, 128 aa, synthetic construct
QVQLQESGPGLVKPSETLSLTCTVSGGSMSNYYWNWIRQPPGKGLEWIGYINYSGVTRYNPSLSSRVTISVDRSKNQFSLKLSSVTAADRAVYYCARSRSCSSDNCYMFWDYYGMDVWGQGTTVTVSS

SEQ ID NO: 75 - protein, 109 aa, synthetic construct
QSVLTQPPSASGTPGQRVTISCSGSTSNIGSNTVNWYKQLPGTAPKLLIYSNDQRPSGVPDRFSGSKSGTSASLAISGLQSEDEADYYCAVWDDSLDGVFGGGTKLTVL

SEQ ID NO: 76 - DNA, 385 bp, synthetic construct
  source 1..385: mol_type = other DNA
caggtgcagctgcaggagtcgggcccaggactggtgaagccttcggagaccctgtccctcacctgcactgtctctggtggctccatgagcaattactactggaattggatccggcagcccccagggaagggtctggagtggattgggtatatcaactacagtggggtcaccagatacaacccctccctcagcagtcgagtcaccatttcagtggacaggtcgaagaaccagttctcgctgaagttgagctctgtgaccgctgcggacagggccgtctattactgtgcgagatctcgatcttgcagtagtgacaactgctatatgttttgggactactacggtatggacgtctggggccaggggaccacggtcaccgtctcctcag

SEQ ID NO: 77 - DNA, 328 bp, synthetic construct
  source 1..328: mol_type = other DNA
cagtctgtgctgacgcagccaccctcagcgtctgggacccccgggcagagggtcaccatctcttgttctggaagcacctccaacatcggaagtaatactgtaaactggtataagcagctcccaggaacggctcccaaactcctcatatatagtaatgatcagcggccctcaggggtccctgaccgattctctggctccaagtctggcacctcagcctccctggccatcagtgggctccagtctgaagatgaggctgattattactgtgcagtctgggatgacagcctggatggggtgttcggcggagggaccaagctgaccgtcctag

SEQ ID NO: 78 - DNA, 24 bp, synthetic construct
  source 1..24: mol_type = other DNA
ggattcagtttcagtaactatgac

SEQ ID NO: 79 - DNA, 24 bp, synthetic construct
  source 1..24: mol_type = other DNA
gtttcttttaatgggaatacaaaa

SEQ ID NO: 80 - DNA, 60 bp, synthetic construct
  source 1..60: mol_type = other DNA
gcgtcgggatattgtcatggtggtctgtgtcacatacagaactacttcgctatggacgtc

SEQ ID NO: 81 - DNA, 18 bp, synthetic construct
  source 1..18: mol_type = other DNA
aaattgggggataattat

SEQ ID NO: 82 - DNA, 33 bp, synthetic construct
  source 1..33: mol_type = other DNA
cagacgtgggacagcagcattgcgccctgggtg

SEQ ID NO: 83 - DNA, 24 bp, synthetic construct
  source 1..24: mol_type = other DNA
ggattcatcgtcagtaacaaccac

SEQ ID NO: 84 - DNA, 21 bp, synthetic construct
  source 1..21: mol_type = other DNA
ctttatagtggtggtagcgca

SEQ ID NO: 85 - DNA, 33 bp, synthetic construct
  source 1..33: mol_type = other DNA
gcgagagatcttcacgcgcccgacaaacccaat

SEQ ID NO: 86 - DNA, 21 bp, synthetic construct
  source 1..21: mol_type = other DNA
aggggtcacagcacctacgcc

SEQ ID NO: 87 - DNA, 21 bp, synthetic construct
  source 1..21: mol_type = other DNA
gttaacagtgatggcaggcac

SEQ ID NO: 88 - DNA, 30 bp, synthetic construct
  source 1..30: mol_type = other DNA
cagacctggggcactgacattcatgtggtg

SEQ ID NO: 89 - DNA, 24 bp, synthetic construct
  source 1..24: mol_type = other DNA
ggatacaagtttgccacctactgg

SEQ ID NO: 90 - DNA, 24 bp, synthetic construct
  source 1..24: mol_type = other DNA
atctatcctgatgactctgatacc

SEQ ID NO: 91 - DNA, 48 bp, synthetic construct
  source 1..48: mol_type = other DNA
gcgagatgctacgatttttggagtggttatcagttcggtatggacgtc

SEQ ID NO: 92 - DNA, 33 bp, synthetic construct
  source 1..33: mol_type = other DNA
cagagcctccggcatgataatggatacaactat

SEQ ID NO: 93 - DNA, 30 bp, synthetic construct
  source 1..30: mol_type = other DNA
atgcaaactctacaaactctgatgttcact

SEQ ID NO: 94 - protein, 7 aa, synthetic construct
GGSISSY

SEQ ID NO: 95 - protein, 5 aa, synthetic construct
YYSGS

SEQ ID NO: 96 - protein, 9 aa, synthetic construct
DYYQLPMDV

SEQ ID NO: 97 - protein, 13 aa, synthetic construct
SGSSSNIGSNYVY

SEQ ID NO: 98 - protein, 7 aa, synthetic construct
RNNQRPS

SEQ ID NO: 99 - protein, 11 aa, synthetic construct
AAWDDSLSGRV

SEQ ID NO: 100 - protein, 121 aa, Human metapneumovirus
LLITPQHGLKESYLEESCSTITEGYLSVLRTGWYTNVFTLEVGDVENLTCADGPSLIKTELDLTKSALRELRTVSADQLAREEQIENPRQSRFVLGAIALGVATAAAVTAGVAIAKTIRLE

SEQ ID NO: 101 - protein, 348 aa, Human metapneumovirus
AILNESQYTIKRDVGTTTAVTPSSLQQEITLLCGEILYAKHADYKYAAEIGIQYISTALGSERVQQILRNSGSEVQVVLTRTYSLGKVKNNKGEDLQMLDIHGVEKSWVEEIDKEARKTMATLLKESSGNIPQNQRPSAPDTPIILLCVGALIFTKLASTIEVGLETTVRRANRVLSDALKRYPRMDIPKIARSFYDLFEQKVYHRSLFIEYGKALGSSSTGSKAESLFVNIFMQAYGAGQTMLRWGVIARSSNNIMLGHVSVQAELKQVTEVYDLVREMGPESGLLHLRQSPKAGLLSLANCPNFASVVLGNASGLGIIGMYRGRVPNTELFSAAESYAKSLKESNK